(19) United States
(12) Patent Application Publication
(10) Pub. No.: US 2026/0297113 A1
(43) Pub. Date: Oct. 1, 2026

(54) DIAZABICYCLOOCTANE INHIBITORS OF KRAS(G12D) AND USES

(71) Applicant: Ranok Therapeutics (Hangzhou) Co. Ltd., Zhejiang (CN)

(72) Inventors: Chenghao Ying, Shanghai (CN); Guoqiang Wang, Hangzhou, Zhejiang (CN); Zhiyong Wang, Hangzhou, Zhejiang (CN); Zimo Yang, Lianyungang, Jiangsu (CN); Jinhua Li, Hangzhou, Zhejiang (CN); Yaya Wang, Shanghai (CN); Yan Dai, Hangzhou, Zhejiang (CN); Weiwen Ying, Lexington, MA (US); Kevin P. Foley, Waltham, MA (US)

(21) Appl. No.: 19/478,316

(22) PCT Filed: Apr. 26, 2024
(86) PCT No.: PCT/US2024/026665

§ 371 Date: Oct. 24, 2025

(30) Foreign Application Priority Data
Apr. 26, 2023 (WO) PCT/CN2023/090892

(51) Int. Cl. C07D 519/00; A61K 31/519; A61P 35/00; C07B 59/00

(52) U.S. Cl. CPC C07D 519/00; A61K 31/519; A61P 35/00; C07B 59/002

(57) ABSTRACT

Provided are small molecule inhibitors of the KRAS (G12D) mutant oncoprotein having the structural formula: and pharmaceutically acceptable salts and compositions thereof, which are useful for treating cancers and related conditions.

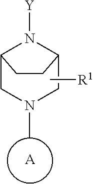

### RELATED APPLICATIONS

This application claims the benefit of priority to international application No. PCT/CN2023/090892, filed Apr. 26, 2023, the entire contents of which are incorporated herein by reference.

### BACKGROUND

Discovered as a human oncogene in the early 1980s, the Kirsten rat sarcoma virus homolog (KRAS) gene encodes a monomeric small 21 kDa GTPase that has long been an elusive cancer drug target (Chang et al., PNAS, 1982, 79:4848-52; McCoy et al., Nature, 1983, 302:79-8). KRAS functions as a molecular switch for promoting cell growth by cycling between GTP- and GDP-bound states. In the GTP-bound state, KRAS signals for growth through the RAF-MAPK and PI3K-AKT-MTOR pathways. KRAS subsequently hydrolyzes GTP to GDP with the aid of GTPase activating proteins (GAPs). This GDP-bound state switches “off” KRAS pro-growth signaling. KRAS can then be switched back “on” by GDP to GTP exchange through the aid of guanine nucleotide exchange factors, such as SOS1 (Cox and Der, Small GTPases, 2010, 1:2-27; Kerk et al., Nat Rev Cancer, 2021, 21:510-525). Preventing this exchange by locking KRAS in the GDP-bound state is a practical method for inhibiting its growth promoting activity.

The human KRAS gene is encoded on Chromosome 12p12.1 and is among the most frequently mutated genes in human cancers (Pylayeva-Gupta et al., Nat Rev Cancer, 2011, 11:761-774). Mutations that prevent GTP-hydrolysis lock KRAS in the active GTP-bound state and reprogram cells for perpetual proliferation. KRAS mutated from glycine (G) at the 12th codon to aspartate (D) creates a chronically active KRAS(G12D) oncoprotein, the gene for which is observed in 6.8% of cancers cases as analyzed by next-generation sequencing (Zhou et al., Pathol Oncol Res, 2020, 26:2835-2837). In tumor type-specific studies, KRAS(G12D) is associated with poor clinical outcomes and observed in 17% of lung, 14.3% of colorectal, and 48% of pancreatic tumors (Aredo et al., Lung Cancer, 2019, 133:144-150; Olmedillas-López et al., World J Gastroenterol, 2017, 23(39):7087-709; Miglio et al., Pathol Res Pract, 2014, 210:307-11; Gou et al., Br J Cancer, 2020, 22:857-867), among other cancers. Historically, oncogenic KRAS mutants have been considered undruggable (McCormick F, Biochem J, 2019, 476:356-74), however the discovery of an allosteric pocket in GDP-bound KRAS has allowed the search for small molecule inhibitors (Ostrem et al., Nature, 2013, 503: 548-51). The G12D mutation moreover provides a unique chemical moiety-binding space due to the encoding of an acidic amino acid residue (D) in place of a small flexible amino acid residue possessing only a hydrogen side chain (G). This alteration of the KRAS protein structure provides a unique space that may be targeted with small molecules drugs that specifically inhibit KRAS(G12D) oncogenic activity. It is therefore desirable to design and develop small molecule drugs that target KRAS(G12D) with sufficient bioavailability to treat diseases such as cancer.

### SUMMARY

Provided herein are small molecule inhibitors of the KRAS(G12D) mutant oncoprotein. Inhibitors of KRAS(G12D) include those having the structural formula I:

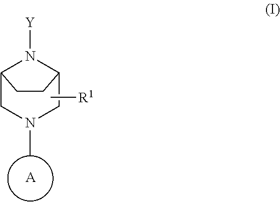

and pharmaceutically acceptable salts and compositions comprising such, wherein R¹, Y, and \textcircled{A} are as defined herein. The use of these compounds, salts, and compositions for treating diseases responsive to the inhibition of KRAS(G12D), such as cancer, is also disclosed.

### DETAILED DESCRIPTION

### 1. General Description of Compounds

As part of a first embodiment, provided is a compound of the Formula I:

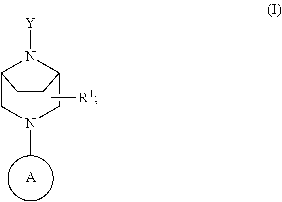

or a pharmaceutically acceptable salt thereof, wherein
Y is CN, C(O)CN, or (C₁-C₄)alkyl optionally substituted with 1 to 3 groups with halo, CN, OH, O(C₁-C₄)alkyl, (C₂-C₄)alkenyl, (C₂-C₄)alkynyl, —COR^{a′}, and —C(O)OR^{a′};
R¹ is hydrogen, halo, OH, (C₁-C₄)alkyl, (C₁-C₄)haloalkyl, (C₁-C₄)hydroxyalkyl, —CHO, —C(O)OR^{b′}, —C(O)ONR^{a′}R^{b′} or a 5- to 6-membered heteroaryl optionally substituted with 1 to 3 groups selected from halo, (C₁-C₄)alkyl, (C₁-C₄)alkoxy, halo(C₁-C₄)alkoxy, and cyano;
R^{a′} and R^{b′} are each independently selected from hydrogen and (C₁-C₄)alkyl, or R^{a′} and R^{b′} are taken together with the nitrogen they attached to form an optionally substituted heterocyclyl; and
\textcircled{A} is optionally substituted heteroaryl.

### 2. Definitions

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein the term “comprising” or “comprises” are used in reference to compositions, methods, and respective component(s) thereof, that are present in a given embodiment, yet open to the inclusion of unspecified elements.

As used herein, the term “alkyl” means a saturated straight chain or branched non-cyclic hydrocarbon having, unless specified otherwise, from 1 to 10 carbon atom e.g., (C₁-C₆)alkyl or (C₁-C₄)alkyl. Representative straight chain alkyls include methyl, ethyl, n-propyl, n-butyl, n-pentyl, n-hexyl, n-heptyl, n-octyl, n-nonyl and n-decyl; while saturated branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like.

As used herein, the term “alkynyl” means a saturated straight chain or branched non-cyclic hydrocarbon having, unless specified otherwise, from 2 to 10 carbon atoms (e.g., (C₂-C₆)alkynyl or (C₂-C₄)alkynyl) and having at least one carbon-carbon triple bond. Representative straight chain and branched alkynyls include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl, and the like.

The term “oxo” refers to the group ═O.

As used herein, the term “haloalkyl” means and alkyl group in which one or more (including all) the hydrogen radicals are replaced by a halo group, wherein each halo group is independently selected from —F, —Cl, —Br, and —I. Representative haloalkyl groups include trifluoromethyl, bromomethyl, 1,2-dichloroethyl, 4-iodobutyl, 2-fluoropentyl, and the like.

“Alkoxy” means an alkyl radical attached through an oxygen linking atom, represented by —O-alkyl. For example, “(C₁-C₄)alkoxy” includes methoxy, ethoxy, proproxy, and butoxy.

“Haloalkoxy” is a haloalkyl group which is attached to another moiety via an oxygen atom such as, e.g., —OCHF₂ or —OCF₃.

As used herein, the term “halogen” or “halo” means F, Cl, Br or I.

As used herein, the term “heterocyclyl” means a 4- to 12-membered monocyclic or polycyclic (e.g., a bridged, fused, or spiro bicyclic ring) saturated or partially unsaturated heterocyclic ring containing 1 to 4 heteroatoms independently selected from N, O, and S. The heterocycle may be attached via any heteroatom or carbon atom, as valency permits. Representative heterocycles include morpholinyl, thiomorpholinyl, pyrrolidinonyl, pyrrolidinyl, piperidinyl, piperazinyl, oxiranyl, dioxanyl, oxetanyl, dihydrofuranyl, dihydropyranyl, isoindolinyl, dihydropyridinyl, tetrahydrofuranyl, tetrahydropyranyl, tetrahydropyrindinyl, tetrahydropyrimidinyl, diazabicyclooctanyl, hexahydropyrrolizinyl, 2-azaspiro[3.3]heptanyl, 2,7-diazaspiro[3.5]nonanyl, 2-azaspiro[3.5]nonanyl, 3-azabicyclo[3.1.0]hexanyl, 2-azabicyclo[3.1.0]hexanyl, 8-azabicyclo[3.2.1]octanyl, 3,8-diazabicyclo[3.2.1]octanyl, 3,6-diazabicyclo[3.1.1]heptanyl, octahydro-1H-pyrrolo[2,3-c]pyridinyl, 1,4-dioxa-8-azaspiro[4.5]decanyl, or 1,2,3,6-tetrahydropyridinyl and the like. Optional substituents on a heterocyclyl group may be present on any substitutable position and, include, e.g., the position at which the heterocyclyl is attached, valence permitting.

The term “spiro” refers to two rings that shares one ring atom (e.g., carbon).

The term “fused” refers to two rings that share two adjacent ring atoms with one another.

The term “bridged” refers to two rings that share three ring atoms with one another.

As used herein, the term “heteroaryl” means a 5- to 12-membered aromatic radical containing 1-4 heteroatoms selected from N, O, and S. A heteroaryl group may be mono- or bicyclic. The heteroaryl may be attached via any heteroatom or carbon atom, as valency permits. Representative heteroaryl groups include pyridyl, furanyl, thienyl, pyrrolyl, oxazolyl, imidazolyl, thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, benzothienyl, and the like. Optional substituents on a heteroaryl group may be present on any substitutable position and, include, e.g., the position at which the heteroaryl is attached, valence permitting.

When used in connection to describe a chemical group that may have multiple points of attachment, a hyphen (-) designates the point of attachment of that group to the variable to which it is defined. For example, —(C₁-C₄)alkylaryl and means that the point of attachment for these groups occurs on the alkyl group.

A hash bond as in “ [symbol] ” represents the point at which the depicted group is attached to the defined variable.

The term “KRAS” refers to the protein product of the KRAS proto-oncogene, GTPase gene.

The term “KRAS(G12D)” refers to the protein product of the KRAS gene carrying a mutation that results in the glycine amino acid at position 12 of KRAS being replaced by an aspartate.

A “chemical entity which binds KRAS^{G12D}” refers to a small molecule or a distinct portion of a larger molecule which binds to a portion of KRAS^{G12D}. In some aspects, the chemical entity which binds KRAS^{G12D} is a small molecule. In some aspects, the chemical entity which binds KRAS^{G12D} is a small molecule having a molecular weight of less than 2,000 g/mol. In some aspects, the chemical entity which binds KRAS^{G12D} induces a confirmation change in KRAS^{G12D}.

The term “SOS1” refers to the protein product of the SOS1 gene that functions as a guanine nucleotide exchange factor for RAS proteins.

The compounds described herein may have chiral centers and/or geometric centers (E- and Z-isomers). It will be understood that the present disclosure encompasses all stereoisomers and geometric isomers. Tautomeric forms of the compounds described herein are also part of the present disclosure.

When the stereochemistry of a disclosed compound is named or depicted by structure, the named or depicted stereoisomer is at least 60%, 70%, 80%, 90%, 99% or 99.9% by weight pure relative to all of the other stereoisomers. Percent by weight pure relative to all of the other stereoisomers is the ratio of the weight of one stereoisomer over the weight of the depicted stereoisomer plus the weight of the other stereoisomers.

For use in medicines, the pharmaceutically acceptable salts of the disclosed compounds refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

As used herein, the term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human and may be referred to as a patient.

As used herein, the terms “treat,” “treating” or “treatment” refer, preferably, to an action to obtain a beneficial or desired clinical result including, but not limited to, alleviation or amelioration of one or more signs or symptoms of a disease or condition, diminishing the extent of disease, stability (i.e., not worsening) of the state of disease, amelioration or palliation of the disease state, diminishing rate of or time to progression, and remission (whether partial or total). “Treatment” can also mean prolonging survival as compared to expected survival in the absence of treatment. Treatment does not need to be curative.

A “therapeutically effective amount” is that amount sufficient to treat a disease in a subject. A therapeutically effective amount can be administered in one or more administrations. In one aspect, a therapeutically effective amount refers to a dosage of from about 0.01 to about 100 mg/kg body weight/day.

The terms “administer,” “administering” or “administration” include any method of delivery of a pharmaceutical composition or agent into a subject's system or to a particular region in or on a subject. In certain embodiments, an agent is administered intravenously, intramuscularly, subcutaneously, intradermally, intranasally, orally, transcutaneously, or mucosally. In certain embodiments, an agent is administered intravenously. In certain embodiments, an agent is administered orally. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, etc.

### 3. Compounds

As part of a second embodiment, the compound of Formula I is of the Formula II:

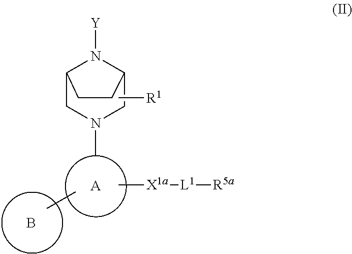

or a pharmaceutically acceptable salt thereof, wherein
X^{1a} is selected from S, O, or —NR^{a};
L¹ is optionally substituted (C₁-C₆)alkylene;
R^{5a} is selected from 5- to 10-membered heterocyclyl and —NR′R″;
R′ and R″ are each independently selected from hydrogen and optionally substituted (C₁-C₄)alkyl, or R′ and R″ are taken together with the nitrogen they attached to form an optionally substituted heterocyclyl; and
\textcircled{B} is an optionally substituted aryl or optionally substituted 5- to 10-membered heteroaryl, and wherein the remaining variables are as described above for Formula I.

As part of a third embodiment, the compound of Formula I is of the Formula III:

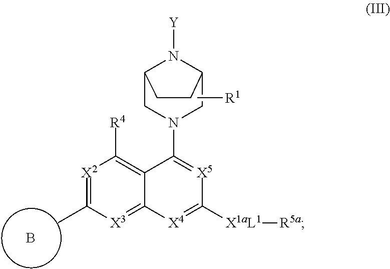

or a pharmaceutically acceptable salt thereof, wherein
X², X³, X⁴, X⁵ are each independently selected from N and CR³; and
R³ and R⁴ are independently selected from hydrogen, (C₁-C₄)alkyl, (C₁-C₄)haloalkyl, (C₁-C₄)alkoxy, deuterated(C₁-C₄)alkoxy, (C₁-C₄)haloalkoxy, (C₁-C₄)alkynyl, (C₁-C₄)alkenyl, halo, (C₃-C₆)cycloalkyl, —O(C₃-C₆)cycloalkyl, cyano, NH₂, —NH(C₁-C₄)alkyl, —N[(C₁-C₄)alkyl]₂, —P(O)[(C₁-C₄)alkyl]₂, and —S(C₁-C₄)alkyl, wherein said (C₃-C₆)cycloalkyl and said (C₃-C₆)cycloalkyl of —O(C₃-C₆)cycloalkyl are optionally substituted with 1 to 3 groups selected from halo, (C₁-C₄)alkyl, (C₁-C₄)alkoxy, halo(C₁-C₄)alkoxy, and cyano wherein the remaining variables are as described above for Formula I.

As part of a fourth embodiment, in the compound of Formula III, X², X⁴ and X⁵ is N; and X³ is CF, wherein the remaining variables are as described above for Formula I or the Formula III of the third embodiment. Alternatively, as part of a fourth embodiment, in the compound of Formula III, X² is CR³, X³ is CF, and X⁴ and X⁵ is N, wherein the remaining variables are as described above for Formula I or the Formula III of the third embodiment. In another alternative, as part of a fourth embodiment, in the compound of Formula III, X² is CR³ and X³, X⁴, and X⁵ is N, wherein the remaining variables are as described above for Formula I or the Formula III of the third embodiment. In yet another alternative, as part of a fourth embodiment, in the compound of Formula III, X², X³, X⁴ and X⁵ is N, wherein the remaining variables are as described above for Formula I or the Formula III of the third embodiment. In yet another alternative, as part of a fourth embodiment, in the compound of Formula III, X², X³, and X⁴ is N; and X⁵ is CR³, wherein the remaining variables are as described above for Formula I or the Formula III of the third embodiment. In yet another alternative, as part of a fourth embodiment, in the compound of Formula III, X², X³ and X⁵ is N; and X⁴ is CR³, wherein the remaining variables are as described above for Formula I or the Formula III of the third embodiment. In yet another alternative, as part of a fourth embodiment, in the compound of Formula III, X² and X⁵ is N, X³ is CF, and X⁴ is CR³, wherein the remaining variables are as described above for Formula I or the Formula III of the third embodiment. In yet another alternative, as part of a fourth embodiment, in the compound of Formula III, X² and X⁴ is N, X³ is CF, and X⁵ is CR³, wherein the remaining variables are as described above for Formula I or the Formula III of the third embodiment.

As part of a fifth embodiment, in the compound of Formula III, R³ is selected from hydrogen, F, Cl, CN, methyl, CF₃, ethyl, and cyclopropyl, wherein the remaining variables are as described above for Formula I or the Formula III of the third or fourth embodiment.

As part of a sixth embodiment, in the compound of Formula III, X¹ is oxygen, wherein the remaining variables are as described above for Formula I or the Formula III of any one of the third to fifth embodiments. Alternatively, as part of a sixth embodiment, in the compound of Formula III, X¹ is sulfur, wherein the remaining variables are as described above for Formula I or the Formula III of any one of the third to fifth embodiments. In another alternative, as part of a sixth embodiment, in the compound of Formula III, X¹ is NMe, wherein the remaining variables are as described above for Formula I or the Formula M of any one of the third to fifth embodiments.

As part of a seventh embodiment, in the compound of Formula III, R⁴ is selected from hydrogen, F, Me, CF₃, CHF₂, cyclopropyl, OMe, OEt, OiPr, OCD₃, OCF₃,

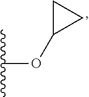 [SMILES: *OC1CC1]

NH₂, NHMe, NMe₂, and SMe, wherein the remaining variables are as described above for Formula I or the Formula III of any one of the third to sixth embodiments.

As part of an eighth embodiment, in the compound of Formula III, L¹ is selected from —CH₂—, —CH₂CH₂—, —CH₂CH₂CH₂—, —CD₂-,

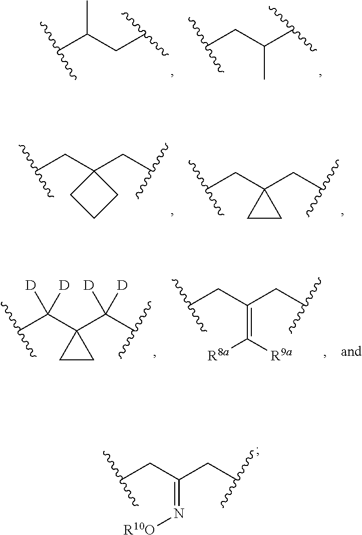

R^{8a} and R^{9a} are each independently selected from hydrogen, halo, CN, (C₁-C₄)alkyl, (C₃-C₆)cycloalkyl, aryl, heteroaryl or R^{8a} and R^{9a} are taken together with the carbon they attached to form cycloalkyl or heterocyclyl; and R¹⁰ is selected from hydrogen, (C₁-C₄)alkyl, (C₃-C₆)cycloalkyl, aryl, heteroaryl, wherein the remaining variables are as described above for Formula I or the Formula III of any one of the third to seventh embodiments.

As part of a ninth embodiment, in the compound of Formula III, R^{5a} is selected from

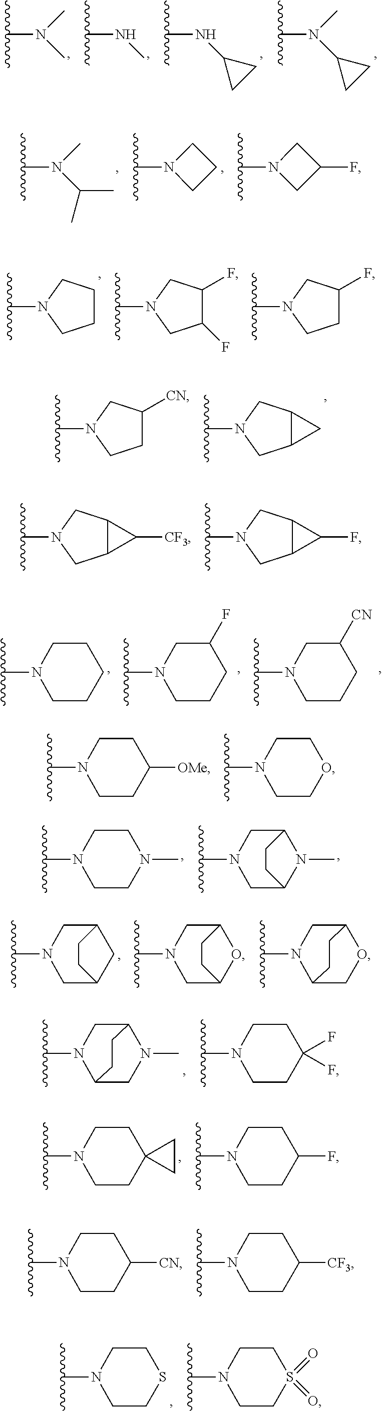

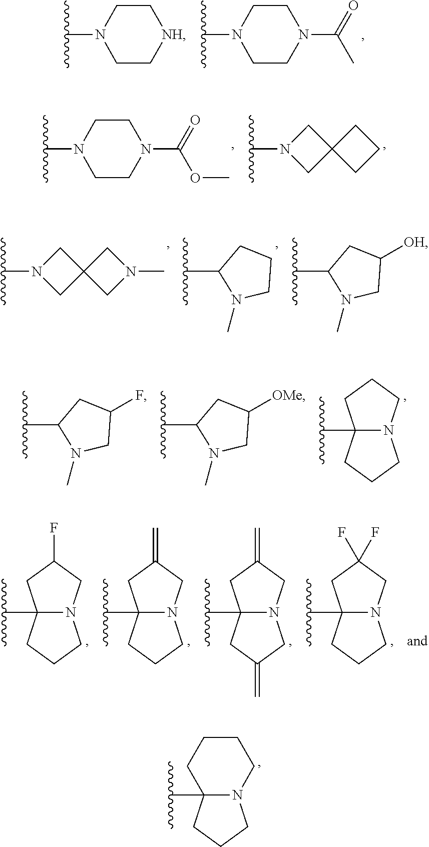

wherein the remaining variables are as described above for Formula I or the Formula III of any one of the third to eighth embodiments.

As part of a tenth embodiment, in the compound of Formula III, R³ is selected from

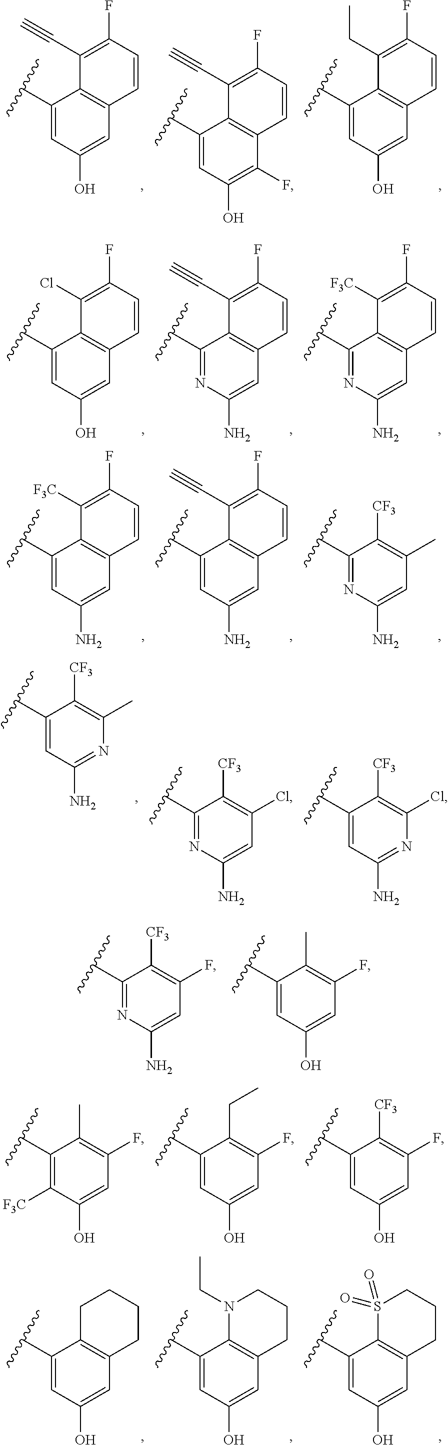

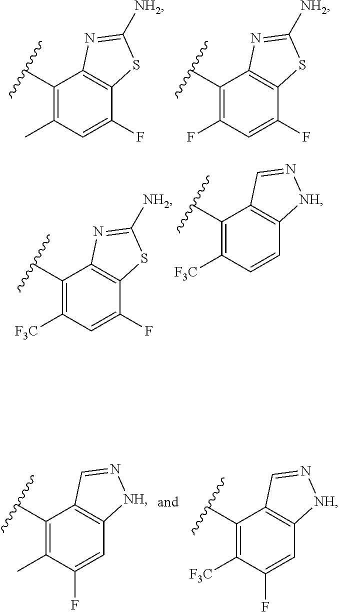

wherein the remaining variables are as described above for Formula I or the Formula III of any one of the third to ninth embodiments.

As part of an eleventh embodiment, the compound of Formula I, II, or III is of the Formula III^{a}:

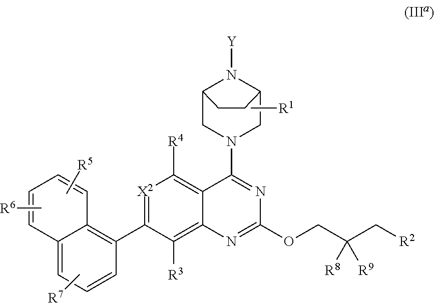

or a pharmaceutically acceptable salt thereof, wherein
X² is CH or N;
R² is a 4- to 6-membered monocyclic heterocyclyl substituted with 1 to 3 groups selected from R^{e} or a 6- to 10-membered bicyclic heterocyclyl optionally substituted with 1 to 3 groups selected from R^{d};
R⁵ is (C₂-C₄)alkynyl;
R⁶ is hydrogen or halo;
R⁷ is hydrogen or OH;
R⁸ and R⁹ are taken together with the carbon they attached to form

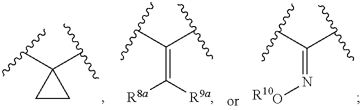

R^{8a} and R^{9a} are each independently selected from hydrogen, halo, (C₁-C₄)alkyl, (C₁-C₄)haloalkyl, (C₁-C₄)alkoxy, deuterated(C₁-C₄)alkoxy, and (C₁-C₄)haloalkoxy; or R^{8a} and R^{9a} are taken together with the carbon they are attached to form (C₃-C₆)cycloalkyl or 4- to 8-membered monocyclic heterocyclyl, wherein said (C₃-C₆)cycloalkyl and said 4- to 8-membered monocyclic heterocyclyl are optionally substituted with 1 to 3 groups selected from halo, (C₁-C₄)alkyl, (C₁-C₄)alkoxy, halo(C₁-C₄)alkoxy, and cyano;
R¹⁰ is selected from hydrogen and optionally substituted (C₁-C₄)alkyl;
R^{a} and R^{b} are each independently selected from hydrogen and (C₁-C₄)alkyl; and
R^{c} and R^{d} are each independently selected from halo, (C₁-C₄)alkyl, (C₁-C₄)haloalkyl, (C₁-C₄)alkoxy, (C₁-C₄)haloalkoxy, cyano, OH, oxo, —C(O)OR^{a}, —C(O)R^{a}, —SO₂R^{a}, —S(O)R^{a}, —SO₂NR^{a}R^{b}, —NR^{a}C(O)R^{b}, —NR^{a}SO₂R^{b}, —NR^{a}R^{b}, and NO, wherein the remaining variables are as described above for Formula I.

As part of a twelfth embodiment, the compound of Formula III^{a} is of the Formula IV:

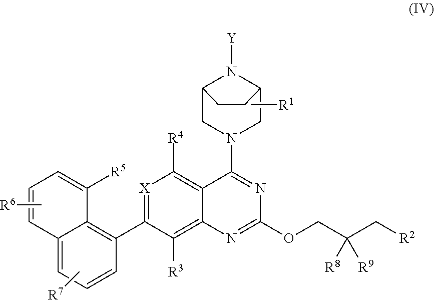

or a pharmaceutically acceptable salt thereof, wherein the variables are as described above for any one of Formula I or III^{a}. Alternatively, as part of a twelfth embodiment, the compound of Formula III is of the Formula V:

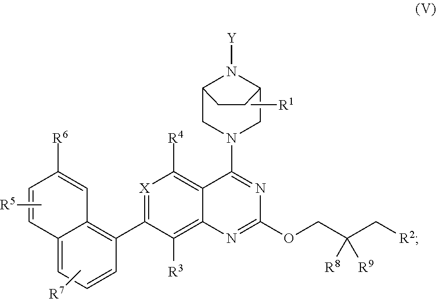

or a pharmaceutically acceptable salt thereof, wherein the variables are as described above for any one of Formula I or III^{a}. In another alternative, as part of a twelfth embodiment, the compound of Formula III is of the Formula VI:

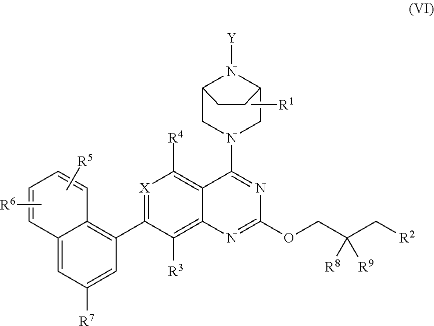

or a pharmaceutically acceptable salt thereof, wherein the variables are as described above for any one of Formula I or III^{a}. In yet another alternative, as part of a twelfth embodiment, the compound of Formula III is of the Formula VII:

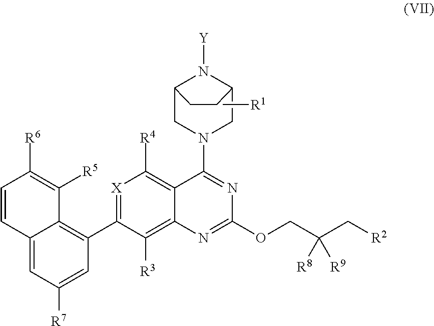

or a pharmaceutically acceptable salt thereof, wherein the variables are as described above for any one of Formula I or III^{a}.

As part of a thirteenth embodiment, R¹ in the compound of Formula I to VII or a pharmaceutically acceptable salt thereof is hydrogen, wherein the remaining variables are described above for any one of Formula I to VII and the first to twelfth embodiments.

As part of a fourteenth embodiment, X in the compound of Formula III^{a} or a pharmaceutically acceptable salt thereof is N, wherein the remaining variables are described above for any one of Formula III^{a} to VII, or the thirteenth embodiment.

As part of a fifteenth embodiment, R³ in the compound of Formula III^{a} or a pharmaceutically acceptable salt thereof is halo, wherein the remaining variables are described above for any one of Formula III^{a} to VII, or the thirteenth or fourteenth embodiment. Alternatively, as part of a fifteenth embodiment, R³ in the compound of Formula III or a pharmaceutically acceptable salt thereof is fluoro, wherein the remaining variables are described above for any one of Formula III^{a} to VII, or the thirteenth or fourteenth embodiment.

As part of a sixteenth embodiment, R⁵ in the compound of Formula III^{a} or a pharmaceutically acceptable salt thereof is (C₂)alkynyl, wherein the remaining variables are described above for any one of Formula III^{a} to VII, or any one of the thirteenth to fifteenth embodiments.

As part of a seventeenth embodiment, R⁶ in the compound of Formula III^{a} or a pharmaceutically acceptable salt thereof is halo, wherein the remaining variables are described above for any one of Formula III^{a} to VII, or any one of the thirteenth to sixteenth embodiments. Alternatively, as part of a ninth embodiment, R⁶ in the compound of Formula III^{a} or a pharmaceutically acceptable salt thereof is fluoro, wherein the remaining variables are described above for any one of Formula III^{a} to VII, or any one of the thirteenth to sixteenth embodiments.

As part of an eighteenth embodiment, R⁷ in the compound of Formula III^{a} or a pharmaceutically acceptable salt thereof is OH, wherein the remaining variables are described above for any one of Formula III^{a} to VII, or any one of the thirteenth to seventeenth embodiments.

As part of a nineteenth embodiment, R⁴ in the compound of Formula III^{a} or a pharmaceutically acceptable salt thereof is selected from hydrogen, (C₁-C₄)alkoxy, deuterated(C₁-C₄)alkoxy, —N[(C₁-C₄)alkyl]₂, halo, (C₃-C₆)cycloalkyl, (C₁-C₄)haloalkoxy, (C₁-C₄)alkyl, and NH₂, wherein the remaining variables are described above for any one of Formula III^{a} to VII, or any one of the thirteenth to eighteenth embodiments. Alternatively, as part of a nineteenth embodiment, R⁴ in the compound of any one of Formula III^{a} or a pharmaceutically acceptable salt thereof is selected from hydrogen, methyl, methoxy, isopropoxy, OCD₃, OCDF₂, OCHF₂, —N(CH₃)₂, NH₂, chloro, and cyclopropyl, wherein the remaining variables are described above for any one of Formula III^{a} to VII, or any one of the thirteenth to eighteenth embodiments. In another alternative, as part of a nineteenth embodiment, R⁴ in the compound of any one of Formula III^{a} or a pharmaceutically acceptable salt thereof is selected from methoxy and OCD, wherein the remaining variables are described above for any one of Formula III^{a} to VII, or any one of the thirteenth to eighteenth embodiments.

As part of a twentieth embodiment, R² in the compound of Formula III^{a} or a pharmaceutically acceptable salt thereof is a 4- to 6-membered nitrogen containing monocyclic heterocyclyl substituted with 1 to 3 groups selected from R^{c} or a 7- to 10-membered nitrogen containing fused or spiro bicyclic heterocyclyl optionally substituted with 1 to 3 groups selected from R^{d}, wherein the remaining variables are described above for any one of Formula III^{a} to VII, or any one of the thirteenth to nineteenth embodiments. Alternatively, as part of a twentieth embodiment, R² in the compound of any one of Formula III^{a} or a pharmaceutically acceptable salt thereof is azetidinyl, piperidinyl, morpholinyl, or pyrrolidinyl, each of which being substituted with 1 to 3 groups selected from R^{c} or R² is 3-azabicyclo[3.1.0]hexanyl, 2-azabicyclo[3.1.0]hexanyl, 1,4-dioxa-8-azaspiro[4.5]decanyl, hexahydro-1H-pyrrolizinyl, or 1,2,3,6-tetrahydropyridinyl, each of which being optionally substituted with 1 to 3 groups selected from R^{d}, wherein the remaining variables are described above for any one of Formula III^{a} to VII, or any one of the thirteenth to nineteenth embodiments. In another alternative, as part of a twentieth embodiment, R² in the compound of any one of Formula III^{a} or a pharmaceutically acceptable salt thereof is selected from 3-azabicyclo[3.1.0]hexanyl, piperidinyl, and hexahydro-1H-pyrrolizinyl, wherein said piperidinyl is optionally substituted with 1 to 3 groups selected from R^{c} and wherein said 3-azabicyclo[3.1.0]hexanyl and said hexahydro-1H-pyrrolizinyl are each optionally substituted with 1 to 3 groups selected from R^{d}, wherein the remaining variables are described above for any one of Formula III^{a} to VII, or any one of the thirteenth to nineteenth embodiments.

As part of a twenty-first embodiment, R^{c} and R^{d} in the compound of any one of Formula III^{a} or a pharmaceutically acceptable salt thereof are each independently selected from halo, cyano, (C₁-C₄)haloalkyl, (C₁-C₄)alkoxy, (C₁-C₄)alkyl, (C₁-C₄)haloalkoxy, —S(O)R^{a}, and —SO₂NR^{a}R^{b}, wherein the remaining variables are described above for any one of Formula III^{a} to VII, or any one of the thirteenth to twentieth embodiments. In another alternative, as part of a twenty-first embodiment, R^{c} and R^{d} in the compound of any one of Formula III^{a} or a pharmaceutically acceptable salt thereof are each independently selected from fluoro, cyano, CF₃, methoxy, isopropyl, OCF₃, —S(O)CH₃, and —SO₂N(CH₃)₂, wherein the remaining variables are described above for any one of Formula III^{a} to VII, or any one of the thirteenth to twentieth embodiments. In yet another alternative, as part of a twenty-first embodiment, R^{c} and R^{d} in the compound of any one of Formula III or a pharmaceutically acceptable salt thereof are each fluoro, wherein the remaining variables are described above for any one of Formula III^{a} to VII, or any one of the thirteenth to twentieth embodiments.

As part of a twenty-second embodiment, R⁸ and R⁹ in the compound of any one of Formula III^{a} or a pharmaceutically acceptable salt thereof are taken together to form

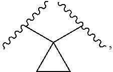 [SMILES: *C1(*)CC1]

wherein the remaining variables are described above for any one of Formula III^{a} to VII, or any one of the thirteenth to twenty-first embodiments.

As part of a twenty-third embodiment, Y in the compound of any one of Formula I to VII or a pharmaceutically acceptable salt thereof is selected from CN, C(O)CN, or (C₁-C₄)alkyl substituted with halo, CN, OH, O(C₁-C₄)alkyl, —COR^{a′}, or —C(O)OR^{a′}, wherein the remaining variables are described above for any one of Formula I to VII, or any one of the second to twenty-second embodiments. Alternatively, as part of a twenty-third embodiment, Y in the compound of any one of Formula I to VII or a pharmaceutically acceptable salt thereof is selected from CN, C(O)CN, CH₂CN, (CH₂)₂CN, CH₂CF₃, (CH₂)₂CH₃, (CH₂)₂OH, (CH₂)₃OH, CH₂C(O)CH₃, CH₂CHCF₂, (CH₂)₃CN, CH(CH₃)(CH₂)₂CN, (CH₂)₂C(O)OH, (CH₂)₂CH₂F, and CH₂C(O)OH, wherein the remaining variables are described above for any one of Formula I to VII, or any one of the second to twenty-second embodiments

Additional compounds are further disclosed in the Exemplification and are included in the present disclosure. Pharmaceutically acceptable salts thereof as well as the neutral forms are included.

### 4. Uses, Formulation and Administration

Compounds and compositions described herein are generally useful as anticancer therapies. In one aspect, the disclosed compounds and compositions behave as inhibitors of KRAS(G12D). Their mechanisms of action include, but are not limited to, inhibiting KRAS(G12D) and thereby impeding down-stream signals that may result in inhibition of cancer cell growth and/or induction of cancer cell death or other KRAS or KRAS(G12D) functions. In one aspect, the disclosed compounds effectuate the inhibition of KRAS(G12D).

Thus, provided herein are methods of treating conditions which are responsive to the inhibition of KRAS(G12D) comprising administering to a subject in need thereof, a therapeutically effective amount of one or more compounds or compositions described herein. Also provided is the use of one or more compounds or compositions described herein in the manufacture of a medicament for treating conditions which are responsive to the inhibition of KRAS(G12D). Further provided is the use of a compound or composition described herein for treating conditions which are responsive to the inhibition of KRAS(G12D).

In one aspect, the condition treated by the present compounds and compositions is a cancer. The terms “cancer” or “tumor” are well known in the art and refer to the presence, e.g., in a subject, of cells possessing characteristics typical of cancer-causing cells, such as uncontrolled proliferation, immortality, metastatic potential, rapid growth and proliferation rate, decreased cell death/apoptosis, and certain characteristic morphological features. Cancer cells are often in the form of a solid tumor. However, cancer also includes non-solid tumors, e.g., blood tumors, e.g., leukemia, wherein the cancer cells are derived from bone marrow. As used herein, the term “cancer” includes pre-malignant as well as malignant cancers. Cancers include, but are not limited to, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, Burkitt's lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin and non-Hodgkin), malignancies and hyperproliferative disorders of the bladder, breast, colon, lung, ovaries, pancreas, prostate, skin, and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor. Other cancers include primary cancer, metastatic cancer, oropharyngeal cancer, hypopharyngeal cancer, liver cancer, gall bladder cancer, bile duct cancer, small intestine cancer, urinary tract cancer, kidney cancer, urothelium cancer, female genital tract cancer, uterine cancer, gestational trophoblastic disease, male genital tract cancer, seminal vesicle cancer, testicular cancer, germ cell tumors, endocrine gland tumors, thyroid cancer, adrenal cancer, pituitary gland cancer, hemangioma, sarcoma arising from bone and soft tissues, Kaposi's sarcoma, nerve cancer, ocular cancer, meningial cancer, glioblastomas, neuromas, neuroblastomas, Schwannomas, solid tumors arising from hematopoietic malignancies such as leukemias, metastatic melanoma, recurrent or persistent ovarian epithelial cancer, fallopian tube cancer, primary peritoneal cancer, gastrointestinal stromal tumors, colorectal cancer, gastric cancer, melanoma, glioblastoma multiforme, non-squamous non-small-cell lung cancer, malignant glioma, epithelial ovarian cancer, primary peritoneal serous cancer, metastatic liver cancer, neuroendocrine carcinoma, refractory malignancy, triple negative breast cancer, HER2-amplified breast cancer, nasopharageal cancer, oral cancer, biliary tract, hepatocellular carcinoma, squamous cell carcinomas of the head and neck (SCCHN), non-medullary thyroid carcinoma, recurrent glioblastoma multiforme, neurofibromatosis type 1, CNS cancer, liposarcoma, leiomyosarcoma, salivary gland cancer, mucosal melanoma, acral/lentiginous melanoma, paraganglioma, pheochromocytoma, advanced metastatic cancer, solid tumor, triple negative breast cancer, colorectal cancer, sarcoma, melanoma, renal carcinoma, endometrial cancer, thyroid cancer, rhabdomyosarcoma, multiple myeloma, ovarian cancer, glioblastoma, gastrointestinal stromal tumor, mantle cell lymphoma, and refractory malignancy.

“Solid tumor,” as used herein, is understood as any pathogenic tumor that can be palpated or detected using imaging methods as an abnormal growth having three dimensions. A solid tumor is differentiated from a blood tumor such as leukemia. However, cells of a blood tumor are derived from bone marrow; therefore, the tissue producing the cancer cells is a solid tissue that can be hypoxic.

“Tumor tissue” or “tumorous tissue” are understood as cells, extracellular matrix, and other naturally occurring components associated with the solid tumor.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the composition.

### EXEMPLIFICATION

### Chemical Synthesis

The representative examples that follow are intended to help illustrate the present disclosure, and are not intended to, nor should they be construed to, limit the scope of the invention. General starting materials used were obtained from commercial sources or prepared in other examples, unless otherwise noted.

### Abbreviations

DMF: dimethylformaldehyde
DCM & CH₂Cl₂: dichloromethane
EtOAc: ethyl acetate
PE: petroleum ether
THF: Tetrahydrofuran
MeOH: Methanol
CDI: 1,1′-Carbonylbis-1H-imidazole
DIEA: N,N-Diisopropylethylamine
HOBt: 1-Hydroxybenzotriazole hydrate
EDCI: 1-Ethyl-3-(3-dimethylaminopropyl)carbodiimide
HATU: 1-[Bis(dimethylamino)methylene]-1H-1,2,3-triazolo[4,5-b]pyridinium 3-oxide hexafluorophosphate
TFA: Trifluoroacetic Acid
DEA: Diethanolamine
DMAP: 4-Dimethylaminopyridine
MTBE: Methyl tert-butyl ether

The compounds described herein were prepared following the procedures outlined in scheme 1.

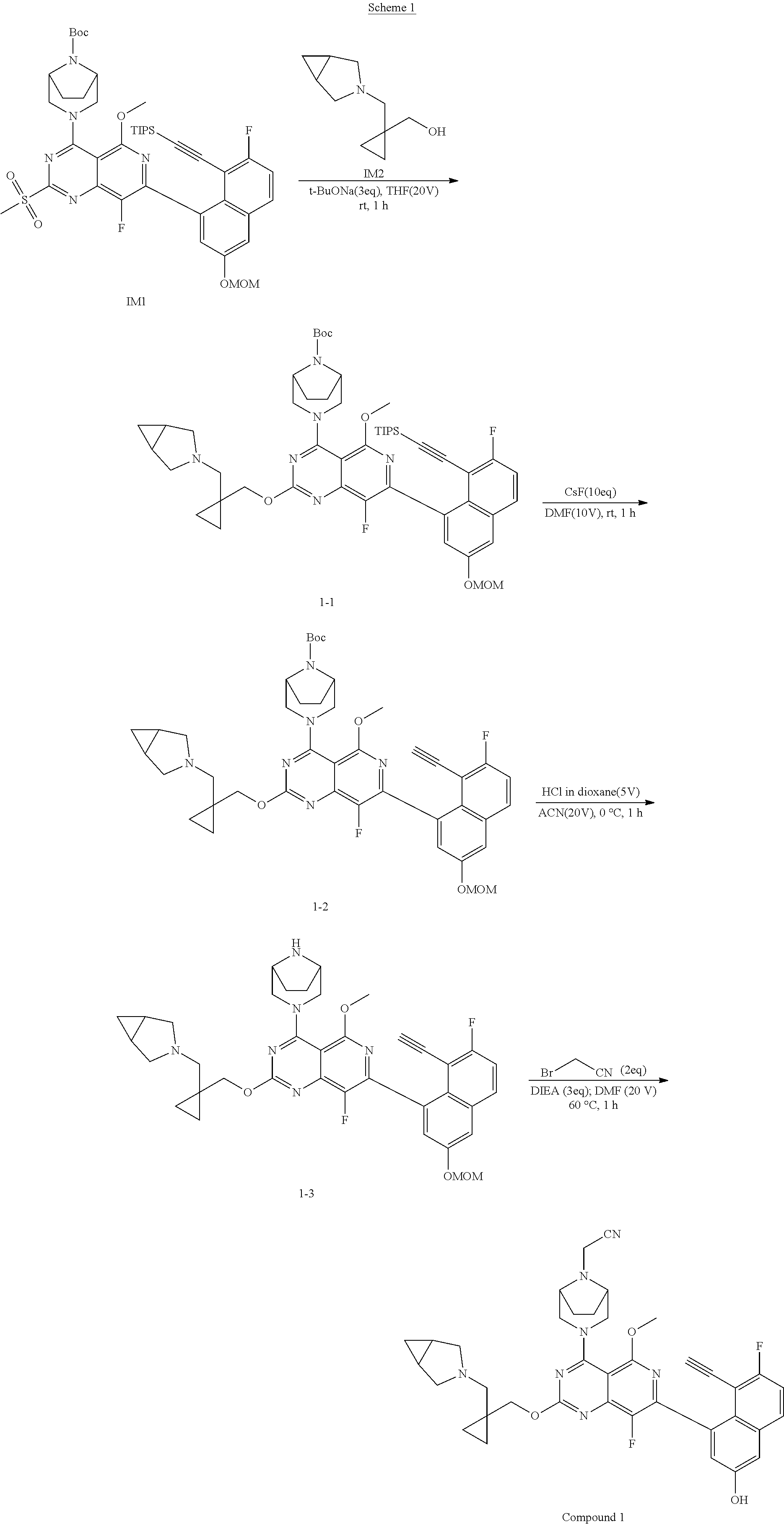

### Preparation of Compound 1

Step 1: tert-butyl 3-{2-[(1-{3-azabicyclo[3.1.0]hexan-3-ylmethyl}cyclopropyl)methoxy]-8-fluoro-7-[7-fluoro-3-(methoxymethoxy)-S-[2-(triisopropylsilyl)ethynyl]naphthalen-1-yl]-5-methoxypyrido[4,3-d]pyrimidin-4-yl}-3,8-diazabicyclo[3.2.1]octane-8-carboxylate. A solution of IM2 (5.01 g, 29.927 mmol, 1.5 equiv, purchased from PharmarBlock) in THF (100 mL) was treated with t-BuONa (5.75 g, 59.853 mmol, 3 equiv) for 30 min at 0° C. under nitrogen atmosphere, followed by the addition of the solution of IM1 (17 g, 19.951 mmol, 1 equiv, purchased from WUXI AppTec) in THF (200 mL) dropwise at 0° C. for 2 h. The reaction was quenched by addition the H₂O (200 mL), The aqueous layer was extracted with DCM (3×200 mL). The residue was purified by silica gel column chromatography, eluted with PE/EA (3:1) to afford 1-1 (11.7 g, 62.44%) as a yellow solid. LCMS: (ES, m/z): 939.2[M+H]⁺.

Step 2: tert-butyl (1R,5S)-3-(2-((1-((3-azabicyclo[3.1.0]hexan-3-yl) methyl)cyclopropyl)methoxy)-7-(8-ethynyl-7-fluoro-3-(methoxymethoxy) naphthalen-1-yl)-8-fluoro-5-methoxypyrido[4,3-d]pyrimidin-4-yl)-3,8-diazabicyclo[3.2.1]octane-8-carboxylate. A solution of 1-1 (11.7 g, 12.457 mmol, 1 equiv) and CsF (18.92 g, 124.570 mmol, 10 equiv) in DMF (200 mL) was stirred for 2 h at room temperature under nitrogen atmosphere. The reaction was quenched with Water at room temperature. The aqueous layer was extracted with EtOAc (3×500 mL). The resulting mixture was concentrated under vacuum. The crude product was used in the next step directly without further purification. LCMS: (ES, m/z): 782.9 [M+H]⁺.

Step 3: 4-{2-[(1-{3-azabicyclo[3.1.0]hexan-3-ylmethyl}cyclopropyl) methoxy]-4-{3,8-diazabicyclo[3.2.1]octan-3-yl}-8-fluoro-5-methoxypyrido[4,3-d]pyrimidin-7-yl}-5-ethynyl-6-fluoronaphthalen-2-ol. A solution of 1-2 in ACN (100 mL) was slowly dropped HCl(gas) in 1,4-dioxane (50 mL, 1645.639 mmol, 143.15 equiv), the solution was stirred for 1 h at 0° C. under nitrogen atmosphere. The mixture was neutralized to pH 10 with NH₃·H₂O. The aqueous layer was extracted with DCM (3×100 mL). The resulting mixture was concentrated under vacuum. The crude product was purified by Prep-HPLC with the following conditions (SHIMADZU): Column, XBridge Shield RP18 OBD Column, 30*150 mm, 5 μm; mobile phase, H₂O (10 mmol/L NH₄HCO₃) and MeOH (60% MeOH up to 80% in 20 min); Detector, uv 254 nm. The 1-3 was obtained as a white solid (3.2 g). ¹H NMR (400 MHz, DMSO-d₆) δ 10.13 (s, 1H), 7.97 (dd, J=9.2, 5.9 Hz, 1H), 7.46 (t, J=9.0 Hz, 1H), 7.38 (d, J=2.6 Hz, 1H), 7.21 (d, J=2.6 Hz, 1H), 4.29-4.17 (m, 2H), 4.13-4.10 (m, 1H), 3.87 (d, J=7.7 Hz, 5H), 3.47 (d, J=6.1 Hz, 2H), 3.42-3.37 (m, 1H), 2.96 (dd, J=8.6, 5.2 Hz, 2H), 2.75-2.62 (m, 1H), 2.42-2.32 (m, 2H), 2.29-2.17 (m, 2H), 1.65-1.55 (m, 4H), 1.35-1.29 (m, 2H), 1.23 (s, 1H), 0.61-0.55 (m, 3H), 0.38 (q, J=4.2 Hz, 2H), 0.29-0.25 (m, 1H). LCMS: (ES, m/z): 638.95 [M+H]⁺.

Step 4: 2-(3-(2-[(1-(3-azabicyclo[3.1.0]hexan-3-ylmethylcyclopropyl) methoxy]-7-(8-ethynyl-7-fluoro-3-hydroxynaphthalen-1-yl)-8-fluoro-5-methoxypyrido[4,3-d]pyrimidin-4-yl-3,8-diazabicyclo[3.2.1]octan-8-yl)acetonitrile. To a stirred mixture of 1-3 (80 mg, 0.125 mmol, 1 equiv) and 2-bromoacetonitrile (30.05 mg, 0.250 mmol, 2 equiv) in DMF (1 mL) was added DIEA (48.56 mg, 0.375 mmol, 3 equiv) dropwise at room temperature under nitrogen atmosphere. The resulting mixture was stirred for 1 h at 60° C. under nitrogen atmosphere. And the reaction was monitored by LC-MS. After the reaction completed, the product was isolated by Pre-HPLC (Column: XBridge BEH Shield RP18 5 m, 30 mm×150 mm; Mobile Phase A: Water (10 mmol/L NH₄HCO₃), Mobile Phase B: ACN; Flow rate: 60 mL/min; Gradient: 55% B to 75% B in 8 min; Wave Length: 254 nm/220 nm; RT1(min): 7.22). This resulted in Compound 1 (17.91 mg, 20.51%) as a yellow solid. ¹H NMR (DMSO-d₆, 400 MHz): δ 10.16 (s, 1H), 7.99 (dd, J=9.2, 5.9 Hz, 1H), 7.48 (t, J=9.0 Hz, 1H), 7.40 (d, J=2.6 Hz, 1H), 7.23 (d, J=2.6 Hz, 1H), 4.23 (q, J=10.8 Hz, 2H), 4.13 (d, J=12.2 Hz, 1H), 3.97 (s, 1H), 3.92 (d, J=9.7 Hz, 4H), 3.58 (s, 2H), 3.43 (d, J=10.0 Hz, 4H), 3.01-2.93 (m, 2H), 2.39 (q, J=12.2 Hz, 2H), 2.24 (d, J=8.4 Hz, 2H), 1.86 (d, J=6.6 Hz, 2H), 1.70-1.51 (m, 2H), 1.36-1.29 (m, 2H), 0.57 (d, J=5.3 Hz, 3H), 0.40 (s, 2H), 0.27 (dq, J=8.0, 3.7 Hz, 1H). LCMS: m/z 677.9 [M+H]⁺.

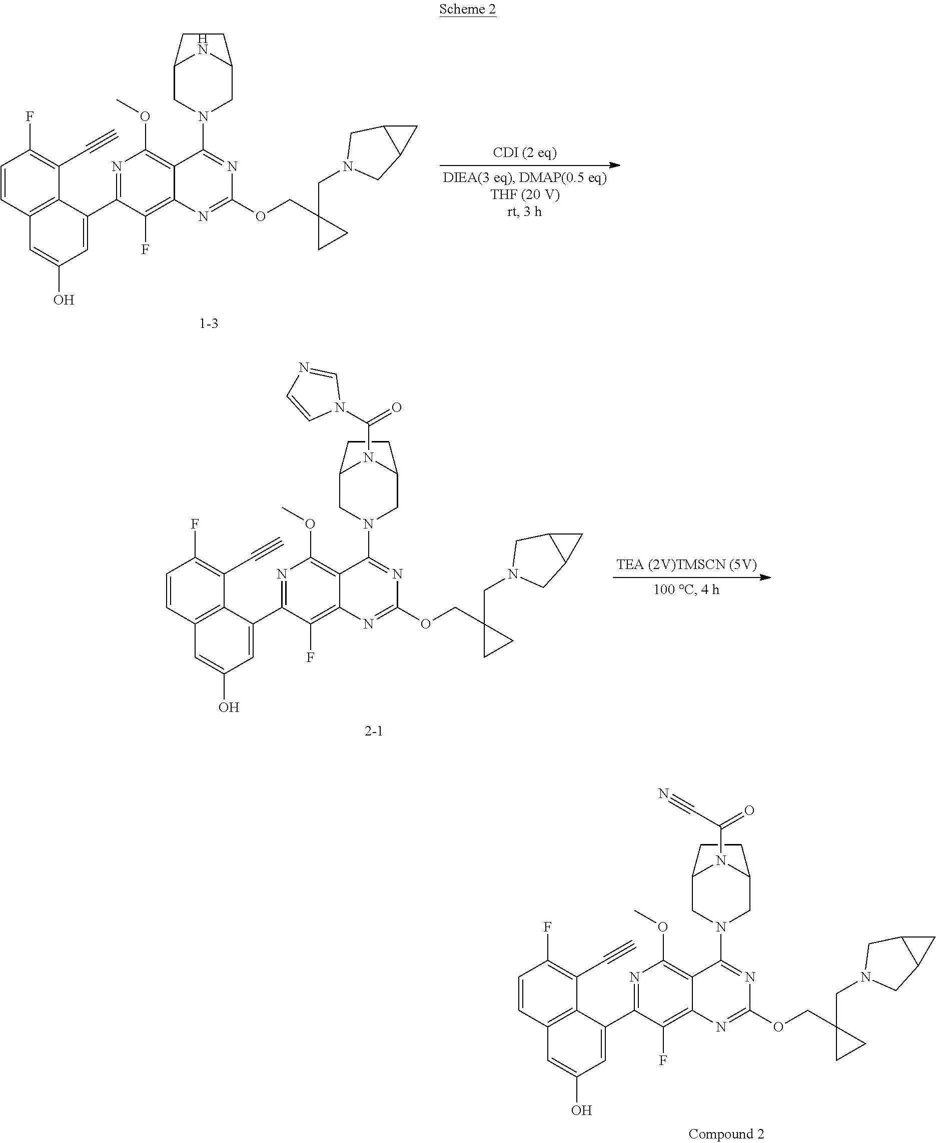 [SMILES: C#Cc1c(F)ccc2cc(O)cc(-c3nc(OC)c4c(N5CC6CCC(C5)N6)nc(OCC5(CN6CC7CC7C6)CC5)nc4c3F)c12 ; C#Cc1c(F)ccc2cc(O)cc(-c3nc(OC)c4c(N5CC6CCC(C5)N6C(=O)n5ccnc5)nc(OCC5(CN6CC7CC7C6)CC5)nc4c3F)c12 ; C#Cc1c(F)ccc2cc(O)cc(-c3nc(OC)c4c(N5CC6CCC(C5)N6C(=O)C#N)nc(OCC5(CN6CC7CC7C6)CC5)nc4c3F)c12]

### Preparation of Compound 2

Step 1: 4-{2-[(1-{3-azabicyclo [3.1.0]hexan-3-ylmethyl} cyclopropyl) methoxy]-8-fluoro-4-[8-(imidazole-1-carbonyl)-3,8-diazabicyclo [3.2.1]octan-3-yl]-5-methoxypyrido[4,3-d]pyrimidin-7-yl}-5-ethynyl-6-fluoronaphthalen-2-ol. To a stirred mixture of 1-3 (190 mg, 0.297 mmol, 1 equiv) and CDI (96.47 mg, 0.594 mmol, 2 equiv) in THF (4 mL) were added DIEA (115.34 mg, 0.891 mmol, 3 equiv) and DMAP (18.17 mg, 0.148 mmol, 0.5 equiv) in portions at room temperature under nitrogen atmosphere. The resulting mixture was stirred for 3 h at room temperature under nitrogen atmosphere. The reaction was monitored by LC-MS. The reaction was quenched with Water at room temperature. The resulting mixture was extracted with DCM (3×5 mL). The combined organic layers were washed with brine (2×5 mL), dried over anhydrous Na₂SO₄. After filtration, the filtrate was concentrated under reduced pressure. The residue was purified by silica gel column chromatography, eluted with DCM/MeOH (10:1) to afford 2-1 (110 mg, 50.46%) as a yellow solid.

LCMS: (ES, m/z): 733[M+H]⁺.

Step 2: 3-{2-[(1-{3-azabicyclo[3.1.0]hexan-3-ylmethyl}cyclopropyl)methoxy]-7-(8-ethynyl-7-fluoro-3-hydroxynaphthalen-1-yl)-8-fluoro-5-methoxypyrido[4,3-d]pyrimidin-4-yl}-3,8-diazabicyclo[3.2.1]octane-8-carbonyl cyanide. A solution of 2-1 (110 mg, 0.150 mmol, 1 equiv) in TMSCN (5 mL) and TEA (2 mL) was stirred for 4 h at 100° C. under nitrogen atmosphere. The mixture was allowed to cool down to room temperature. The reaction was quenched with Water at room temperature. The resulting mixture was extracted with DCM (3×5 mL). The combined organic layers were washed with brine (2×5 mL), dried over anhydrous Na₂SO₄. After filtration, the filtrate was concentrated under reduced pressure. The crude product was purified by Prep-HPLC with the following conditions (Column: XBridge BEH C18 OBD Prep Column 130, 5 m, 30 mm×150 mm; Mobile Phase A: Water (10 mmol/L NH4HCO3), Mobile Phase B: ACN; Flow rate: 60 ml/min mL/min; Gradient: 48% B to 77% B in 10 min; Wave Length: 254 nm/220 nm; RT1(min): 8.88). This resulted in Compound 2 (9.09 mg, 8.22%) as a yellow solid. ¹H NMR (DMSO-d₆, 400 MHz): δ 10.16 (s, 1H), 7.98 (dd, J=9.2, 5.9 Hz, 1H), 7.47 (t, J=9.0 Hz, 1H), 7.39 (d, J=2.6 Hz, 1H), 7.22 (t, J=2.4 Hz, 1H), 4.67 (t, J=13.2 Hz, 1H), 4.63-4.55 (m, 1H), 4.39-4.11 (m, 4H), 4.02-3.81 (m, 4H), 3.64 (t, J=13.1 Hz, 1H), 3.46 (s, 1H), 2.95 (dd, J=8.6, 7.0 Hz, 2H), 2.38 (d, J=14.0 Hz, 2H), 2.23 (d, J=8.5 Hz, 1H), 2.06-1.98 (m, 1H), 1.84 (s, 3H), 1.31 (t, J=4.6 Hz, 2H), 1.24 (s, 1H), 0.63-0.48 (m, 3H), 0.39 (q, J=4.2 Hz, 2H), 0.25 (td, J=7.7, 3.7 Hz, 1H). LCMS: (ES, m/z): 692.1 [M+H]⁺.

The following compounds in Table 1 were prepared according to the methods described above using the appropriate starting materials.

TABLE 1
The structure of Compounds

| | | LCMS: m/z | ¹H NMR |
|---|---|---|---|
| No | Structure | [M + H]⁺ | (DMSO-d₆, 400 MHz) |
| 3 | 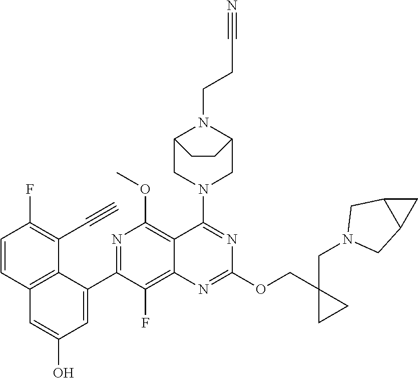 [SMILES: C#Cc1c(F)ccc2cc(O)cc(-c3nc(OC)c4c(N5CC6CCC(C5)N6CCC#N)nc(OCC5(CN6CC7CC7C6)CC5)nc4c3F)c12] | 692.3 | δ 10.15 (s, 1H), 7.90- 8.05 (m, 1H), 7.43-7.51 (m, 1H) 7.38 (d, J = 2.40 Hz, 1H), 7.17-7.24 (m, 1H), 4.17-4.26 (m, 2H), 4.03-4.15 (m, 1H), 3.90 (s, 1H), 3.88 (s, 3H), 3.39 ( s, 3H), 2.95 (dd, J = 8.30, 5.30 Hz, 3H), 2.67-2.71 (m, 2H), 2.57 (d, J = 6.10 Hz, 2H), 2.36 (s, 3H), 2.24 (s, 2H), 1.76-1.82 (m, 2H), 1.48-1.62 (m, 2H), 1.27-1.35 (m, 2H), 0.56 (s, 3H), 0.35-0.41 (m, 2H), 0.21-0.30 (m, 1H). |
| 4 | 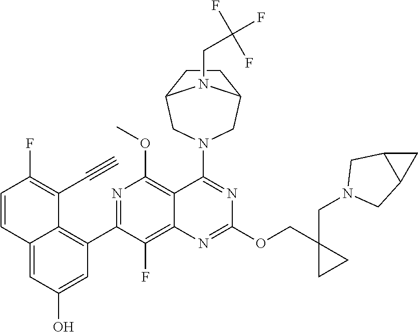 [SMILES: C#Cc1c(F)ccc2cc(O)cc(-c3nc(OC)c4c(N5CC6CCC(C5)N6CC(F)(F)F)nc(OCC5(CN6CC7CC7C6)CC5)nc4c3F)c12] | 721.3 | δ 10.21-10.06 (m, 1H), 8.01-7.92 (m, 1H), 7.50-7.43 (m, 1H), 7.38 (d, J = 2.4 Hz, 1H), 7.23-7.19 (m, 1H), 4.26-4.17 (m, 2H), 4.12-4.01 (m, 1H), 3.91 (s, 2H), 3.89 (s, 3H), 3.44 (d, J = 6.4 Hz, 2H), 3.19-3.11 (m, 2H), 2.96 (dd, J = 5.9, 8.4 Hz, 2H), 2.44-2.31 (m, 4H), 2.23 (d, J = 8.4 Hz, 2H), 1.86-1.73 (m, 2H), 1.66-1.53 (m, 2H), 1.35-1.28 (m, 2H), 0.61-0.54 (m, 3H), 0.38 (s, 2H), 0.29-0.21 (m, 1H). |
| 5 | 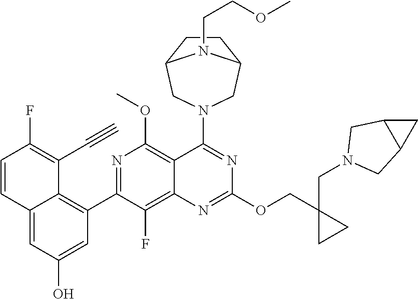 [SMILES: C#Cc1c(F)ccc2cc(O)cc(-c3nc(OC)c4c(N5CC6CCC(C5)N6CCOC)nc(OCC5(CN6CC7CC7C6)CC5)nc4c3F)c12] | 697.3 | δ 10.20-10.08 (m, 1H), 8.01-7.92 (m, 1H), 7.51-7.43 (m, 1H), 7.38 (d, J = 2.4 Hz, 1H), 7.21 (d, J = 2.4 Hz, 1H), 4.26-4.19 (m, 2H), 4.16-4.05 (m, 1H), 3.89 (s, 4H), 3.54-3.40 (m, 7H), 3.33 (s, 3H), 3.28 (s, 3H), 3.24-3.21 (m, 1H), 2.62 (d, J = 1.4 Hz, 4H), 1.85 (s, 2H), 1.65-1.44 (m, 3H), 1.31-1.20 (m, 1H), 0.64 (s, 3H), 0.54-0.34 (m, 3H). |
| 6 | 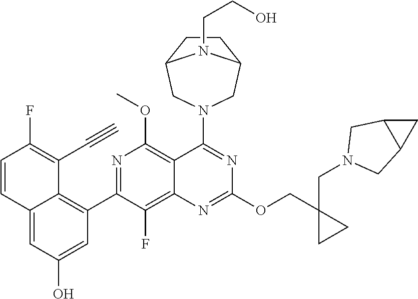 [SMILES: C#Cc1c(F)ccc2cc(O)cc(-c3nc(OC)c4c(N5CC6CCC(C5)N6CCO)nc(OCC5(CN6CC7CC7C6)CC5)nc4c3F)c12] | 683.2 | δ 10.38-9.93 (m, 1H), 8.14 (s, 1H), 7.97 (dd, J = 5.8, 9.2 Hz, 1H), 7.52-7.43 (m, 1H), 7.38 (d, J = 2.4 Hz, 1H), 7.21 (d, J = 2.4 Hz, 1H), 4.26-4.16 (m, 2H), 4.07 (d, J = 9.8 Hz, 1H), 3.88 (s, 3H), 3.55 (t, J = 6.0 Hz, 2H), 3.43 (d, J = 11.2 Hz, 4H), 3.05-2.98 (m, 2H), 2.47-2.42 (m, 2H), 2.33 (d, J = 1.8 Hz, 2H), 1.83 (s, 2H), 1.62-1.49 (m, 2H), 1.35 (d, J = 2.8 Hz, 2H), 0.64-0.53 (m, 3H), 0.41 (s, 2H), 0.30 (dt, J = 3.6, 7.3 Hz, 1H). |
| 7 | 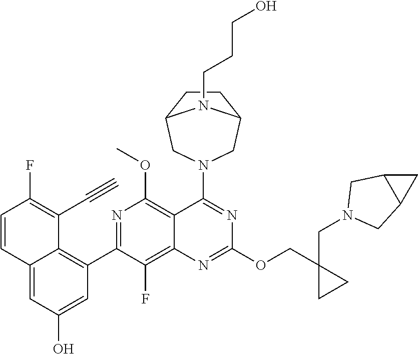 [SMILES: C#Cc1c(F)ccc2cc(O)cc(-c3nc(OC)c4c(N5CC6CCC(C5)N6CCCO)nc(OCC5(CN6CC7CC7C6)CC5)nc4c3F)c12] | 697.4 | δ 10.20-10.05 (m, 1H), 8.13 (s, 1H), 8.00-7.93 (m, 1H), 7.50-7.43 (m, 1H), 7.39 (d, J = 2.4 Hz, 1H), 7.22 (d, J = 2.4 Hz, 1H), 4.27-4.19 (m, 2H), 4.18-4.11 (m, 1H), 4.04-3.95 (m, 1H), 3.93-3.87 (m, 4H), 3.62-3.39 (m, 8H), 3.35-3.27 (m, 2H), 3.09 (d, J = 1.0 Hz, 2H), 2.59 (s, 2H), 1.96-1.83 (m, 2H), 1.75-1.61 (m, 4H), 1.41 (s, 2H), 0.61 (s, 3H), 0.46 (s, 2H), 0.36 (s, 1H). |
| 8 | 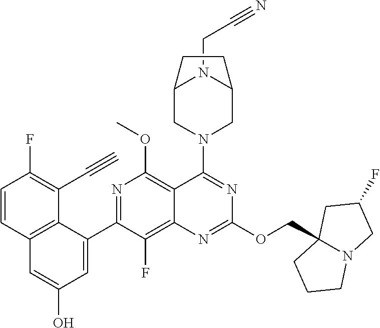 [SMILES: C#Cc1c(F)ccc2cc(O)cc(-c3nc(OC)c4c(N5CC6CCC(C5)N6CC#N)nc(OC[C@]56CCCN5C[C@@H](F)C6)nc4c3F)c12] | 670.3 | δ 8.35 (s, 1H), 7.93- 8.00 (m, 1H), 7.43-7.50 (m, 1H) 7.36-7.41 (m, 1H), 7.22 (s, 1H), 5.16- 5.40 (m, 1H), 4.06-4.15 (m, 2H), 3.97-4.04 (m, 2H), 3.92 (d, J = 3.40 Hz, 1H), 3.89 (s, 3H), 3.55-3.59 (m, 2H), 3.36-3.45 (m, 5H), 3.03-3.12 (m, 2H), 3.01 (s, 1H), 2.78-2.87 (m, 1H), 2.07-2.15 (m, 1H), 1.92-2.07 (m, 2H), 1.68-1.91 (m, 5H), 1.49-1.68 (m, 2H). |
| 9 | 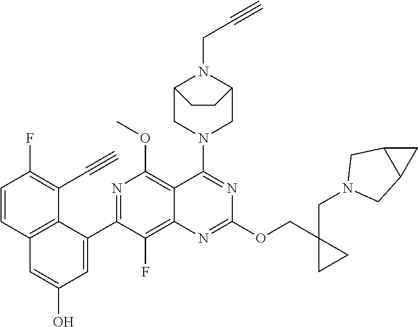 [SMILES: C#CCN1C2CCC1CN(c1nc(OCC3(CN4CC5CC5C4)CC3)nc3c(F)c(-c4cc(O)cc5ccc(F)c(C#C)c45)nc(OC)c13)C2] | 677.4 | δ 10.54-9.86 (m, 1H), 8.17 (s, 1H), 7.97 (dd, J = 6.0, 9.2 Hz, 1H), 7.46 (t, J = 9.0 Hz, 1H), 7.38 (d, J = 2.4 Hz, 1H), 7.21 (d, J = 2.4 Hz, 1H), 4.26-4.16 (m, 2H), 4.16-4.05 (m, 1H), 3.94 (dd, J = 6.8, 9.0 Hz, 1H), 3.90 (s, 1H), 3.88 (s, 2H), 3.40 (d, J = 11.0 Hz, 4H), 3.17 (d, J = 2.0 Hz, 2H), 3.15- 3.14 (m, 1H), 2.96 (dd, J = 6.4, 8.4 Hz, 2H), 2.45-2.31 (m, 3H), 2.23 (d, J = 8.3 Hz, 2H), 1.88-1.75 (m, 2H), 1.63-1.46 (m, 2H), 1.35-1.27 (m, 2H), 0.60-0.53 (m, 3H), 0.41-0.35 (m, 2H), 0.25 (dt, J = 3.6, 7.6 Hz, 1H). |
| 10 | 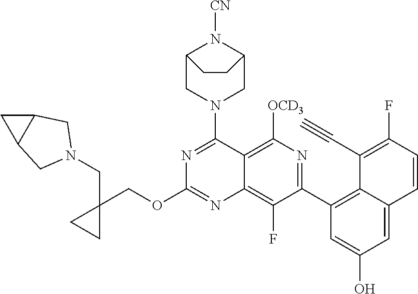 | 667.1 | δ 9.93 (s, 1H), 7.74 (dd, J = 9.2, 5.9 Hz, 1H), 7.23 (t, J = 9.0 Hz, 1H), 7.16 (d, J = 2.6 Hz, 1H), 7.00 (d, J = 2.5 Hz, 1H), 4.05- 3.97 (m, 2H), 3.97- 3.89 (m, 3H), 3.81- 3.76 (m, 1H), 3.75 (d, J = 1.0 Hz, 1H), 3.33- 3.25 (m, 2H), 2.72 (dd, J = 8.6, 5.1 Hz, 2H), 2.20-2.09 (m, 2H), 2.04-1.96 (m, 2H), 1.76-1.60 (m, 3H), 1.55 (d, J = 9.0 Hz, 1H), 1.08 (dt, J = 6.9, 2.9 Hz, 2H), 0.36- 0.29 (m, 3H), 0.16 (q, J = 4.2 Hz, 2H), 0.02 (td, J = 7.6, 3.7 Hz, 1H). |
| 11 | 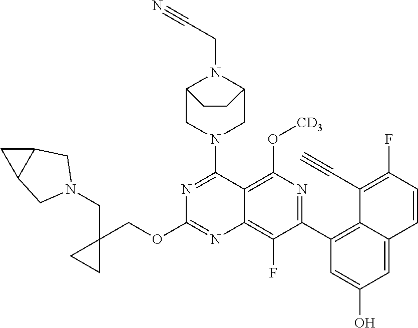 [SMILES: [2H]C([2H])([2H])Oc1nc(-c2cc(O)cc3ccc(F)c(C#C)c23)c(F)c2nc(OCC3(CN4CC5CC5C4)CC3)nc(N3CC4CCC(C3)N4CC#N)c12] | 681.3 | δ 10.13 (s, 1H), 7.97 (dd, J = 9.2, 5.9 Hz, 1H), 7.46 (t, J = 9.0 Hz, 1H), 7.38 (d, J = 2.5 Hz, 1H), 7.21 (d, J = 2.5 Hz, 1H), 4.21 (q, J = 10.8 Hz, 2H), 4.13- 4.11 (m, 1H), 3.97-3.95 (m, 1H), 3.90 (d, J = 1.0 Hz, 1H), 3.56 (s, 2H), 3.39 (s, 4H), 2.95 (dd, J = 8.6, 6.3 Hz, 2H), 2.44-2.29 (m, 2H), 2.23 (d, J = 8.5 Hz, 2H), 1.85 (d, J = 6.5 Hz, 2H), 1.62-1.55 (m, 2H), 1.31 (dd, J = 6.9, 3.4 Hz, 2H), 0.61- 0.51 (m, 3H), 0.41- 0.34 (m, 2H), 0.25 (td, J = 7.6, 3.6 Hz, 1H). |
| 12 | 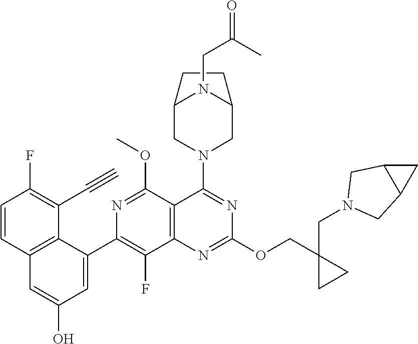 [SMILES: C#Cc1c(F)ccc2cc(O)cc(-c3nc(OC)c4c(N5CC6CCC(C5)N6CC(C)=O)nc(OCC5(CN6CC7CC7C6)CC5)nc4c3F)c12] | 695.5 | δ 10.49-9.83 (m, 1H), 8.25-8.21 (m, 1H), 8.00-7.94 (m, 1H), 7.50-7.43 (m, 1H), 7.38 (d, J = 2.6 Hz, 1H), 7.24-7.20 (m, 1H), 4.24-4.18 (m, 2H), 3.90-3.89 (m, 1H), 3.89 (s, 3H), 3.48-3.42 (m, 4H), 3.23 (s, 2H), 2.98- 2.94 (m, 2H), 2.36 (s, 2H), 2.25-2.22 (m, 3H), 2.17 (s, 3H), 1.82-1.77 (m, 2H), 1.60-1.51 (m, 2H), 1.33-1.30 (m, 2H), 0.62-0.52 (m, 4H), 0.40-0.37 (m, 2H), 0.27 (s, 1H). |
| 13 | 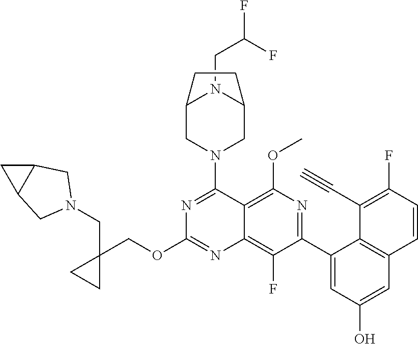 [SMILES: C#Cc1c(F)ccc2cc(O)cc(-c3nc(OC)c4c(N5CC6CCC(C5)N6CC(F)F)nc(OCC5(CN6CC7CC7C6)CC5)nc4c3F)c12] | 703.2 | δ 10.14 (s, 1H), 7.97 (dd, J = 9.2, 5.9 Hz, 1H), 7.46 (t, J = 9.0 Hz, 1H), 7.38 (d, J = 2.6 Hz, 1H), 7.21 (d, J = 2.5 Hz, 1H), 6.10 (tt, J = 56.1, 4.3 Hz, 1H), 4.30-4.12 (m, 2H), 4.06 (s, 1H), 3.89 (d, J = 7.3 Hz, 5H), 3.48- 3.33 (m, 4H), 2.95 (dd, J = 8.6, 5.8 Hz, 2H), 2.76 (td, J = 15.6, 4.4 Hz, 2H), 2.37 (q, J = 12.2 Hz, 2H), 2.23 (d, J = 8.5 Hz, 2H), 1.87- 1.72 (m, 2H), 1.65- 1.49 (m, 2H), 1.36- 1.26 (m, 2H), 0.56 (d, J = 4.1 Hz, 3H), 0.38 (q, J = 4.1 Hz, 2H), 0.26 (td, J = 7.7, 3.7 Hz, 1H). |
| 14 | 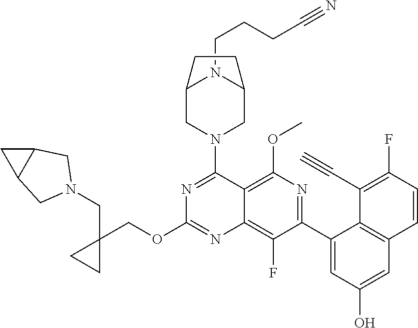 [SMILES: C#Cc1c(F)ccc2cc(O)cc(-c3nc(OC)c4c(N5CC6CCC(C5)N6CCCC#N)nc(OCC5(CN6CC7CC7C6)CC5)nc4c3F)c12] | 705.8 | δ 8.38 (s, 1H), 7.96 (dd, J = 9.00, 6.00 Hz, 1H), 7.46 (t, J = 9.0 Hz, 1H), 7.38 (d, J = 2.4 Hz, 1 H) 7.22 (d, J = 2.4 Hz, 1H), 4.21 (d, J = 6.80 Hz, 1H), 3.88 (s, 3H), 3.18-3.52 (m, 12H), 2.95 (dd, J = 8.4, 5.40 Hz, 2H), 2.65-2.69 (m, 1H), 2.60 (t, J = 7.2 Hz, 1H), 2.35-2.42 (m, 3H), 2.33 (d, J = 1.8 Hz, 1H), 2.23 (d, J = 8.4 Hz, 2H), 1.70-1.81 (m, 3H), 1.47-1.61 (m, 1H), 1.23-1.39 (m, 2H), 0.50-0.62 (m, 2H), 0.35-0.41 (m, 1H), 0.26 (dt, J = 7.6 Hz, 1H). |
| 15 | 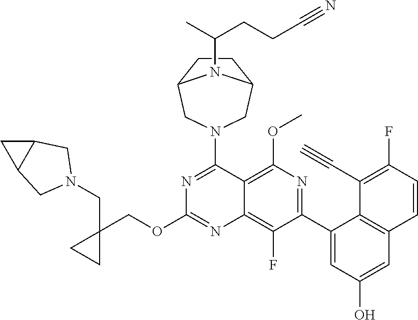 [SMILES: C#Cc1c(F)ccc2cc(O)cc(-c3nc(OC)c4c(N5CC6CCC(C5)N6C(C)CCC#N)nc(OCC5(CN6CC7CC7C6)CC5)nc4c3F)c12] | 720.8 | δ 8.28-8.26 (m, 1H), 7.99-7.94 (m, 1H), 7.49-7.43 (m, 1H), 7.40-7.36 (m, 1H), 7.24-7.20 (m, 1H), 4.23-4.18 (m, 2H), 3.88 (s, 3H), 3.87-3.86 (m, 1H), 3.51 (d, J = 6.8 Hz, 3H), 3.45-3.41 (m, 2H), 2.95 (d, J = 5.6 Hz, 3H), 2.63-2.57 (m, 4H), 2.37 (d, J = 14.6 Hz, 2H), 2.23 (d, J = 8.4 Hz, 2H), 1.82-1.69 (m, 4H), 1.34-1.30 (m, 2H), 1.15 (s, 1H), 1.04- 1.00 (m, 3H), 0.58-0.55 (m, 3H), 0.40-0.36 (m, 2H), 0.25 (d, J = 3.6 Hz, 1H). |
| 16 | 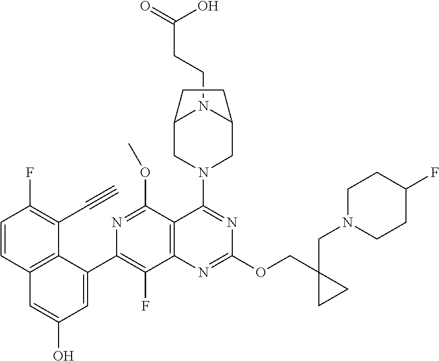 [SMILES: C#Cc1c(F)ccc2cc(O)cc(-c3nc(OC)c4c(N5CC6CCC(C5)N6CCC(=O)O)nc(OCC5(CN6CCC(F)CC6)CC5)nc4c3F)c12] | 731.8 | δ 10.18 (d, J = 4.6 Hz, 1H), 8.27-8.23 (m, 1H), 7.97 (d, J = 6.0, 9.2 Hz, 1H), 7.50-7.43 (m, 1H), 7.38 (d, J = 2.4 Hz, 1H), 7.21 (d, J = 2.6 Hz, 1H), 4.75-4.53 (m, 1H), 4.26 (s, 2H), 4.13- 4.04 (m, 1H), 3.90 (s, 1H), 3.88 (s, 3H), 3.70- 3.47 (m, 3H), 2.62-2.54 (m, 4H), 2.42-2.41 (m, 2H), 2.30 (d, J = 6.0 Hz, 4H), 1.84-1.74 (m, 5H), 1.70-1.53 (m, 5H), 0.64-0.61 (m, 2H), 0.40 (s, 2H). |
| 17 | 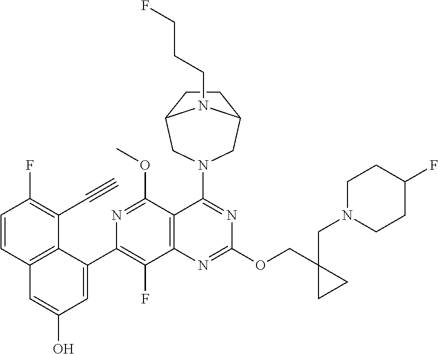 [SMILES: C#Cc1c(F)ccc2cc(O)cc(-c3nc(OC)c4c(N5CC6CCC(C5)N6CCCF)nc(OCC5(CN6CCC(F)CC6)CC5)nc4c3F)c12] | 719.5 | δ 8.43 (s, 1H), 7.76- 7.69 (m, 1H), 7.38 (s, 1H), 7.30 (d, J = 2.6 Hz, 1H), 7.22 (t, J = 8.8 Hz, 1H), 4.84-4.64 (m, 2H), 4.54 (t, J = 5.8 Hz, 1H), 4.45-4.36 (m, 2H), 4.35-4.22 (m, 1H), 4.07-3.95 (m, 1H), 3.93 (s, 3H), 3.70-3.63 (m, 1H), 3.58-3.51 (m, 1H), 3.35 (s, 2H), 3.09-3.02 (m, 1H), 2.95 (s, 1H), 2.90-2.77 (m, 4H), 2.58 (t, J = 7.2 Hz, 2H), 2.27-2.13 (m, 4H), 2.01-1.97 (m, 2H), 1.94-1.88 (m, 4H), 1.76 (s, 1H), 0.82 (s, 2H), 0.67 (s, 2H). |
| 18 | 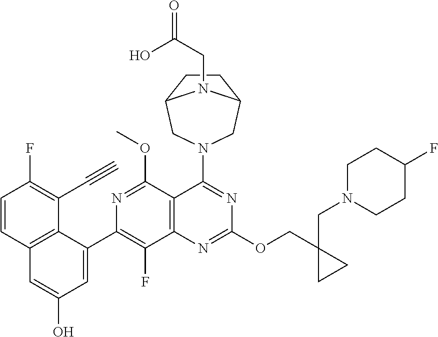 [SMILES: C#Cc1c(F)ccc2cc(O)cc(-c3nc(OC)c4c(N5CC6CCC(C5)N6CC(=O)O)nc(OCC5(CN6CCC(F)CC6)CC5)nc4c3F)c12] | 717.3 | δ 8.14 (s, 1H), 8.00- 7.93 (m, 1H), 7.50-7.42 (m, 1H), 7.38 (d, J = 2.6 Hz, 1H), 7.24-7.19 (m, 1H), 4.73-4.54 (m, 1H), 4.27 (s, 2H), 4.12- 4.04 (m, 1H), 3.92-3.87 (m, 4H), 3.54-3.48 (m, 2H), 3.46 (d, J = 8.0 Hz, 3H), 3.17 (s, 2H), 2.37-2.27 (m, 5H), 1.90-1.73 (m, 5H), 1.69-1.54 (m, 4H), 0.66-0.60 (m, 2H), 0.41 (s, 2H). |

### Biological Assays/Testing

### Cell Lines

The following cancer cell lines were employed: AGS gastric carcinoma [heterozygous G12D](ATCC, CRL-1739); A-427 lung carcinoma [heterozygous G12D](ATCC, HTB-53); ASPC1 pancreatic adenocarcinoma [homozygous G12D](ATCC, CRL-1682) and SW 1990 pancreatic adenocarcinoma [homozygous G12D](ATCC CRL-2172). Cell lines were cultured essentially according to ATCC recommendations.

### Cancer Cell Line Proliferation (CellTiter-Glo® Assays)

AGS, A-427, ASPC1, SW 1990, and GP2D cells were plated in 96-well tissue culture plates at 4,000 cells/well and incubated at 37° G25% CO2 for 72 hr in 100 μl of media. 3-old serial dilutions of each test compound were prepared ranging from 20 μM to 1.02 nM. Each cell line was then treated with test compounds at various concentrations with a final concentration of 0.5% DMSO/well, and then incubated at 37° C./5% CO2 for 72 hr. 100 μl of CellTiter-Glo® Reagent (Promega Corporation, Madison, WI) was added to each well and processed according manufacturer's protocol. Results were analyzed and IC50 values were calculated in GraphPad 7 software. Results are listed in Table 2. AGS proliferation assay: A. EC50<100 nM; B. EC50=100-1000 nM; C. EC50>1000 nM; GP2D proliferation assay: A. EC50<100 nM; B. EC50=100-1000 nM; C. EC50>1000 nM; MKN1 proliferation assay: A. EC50<100 nM; B. EC50=100-1000 nM; C. EC50>1000 nM;

TABLE 2
Cell line proliferation data of the compounds.

| | CTG(IC50) | | |
|---|---|---|---|
| | ABS-72 H(nM) | | |
| Compound | AGS | GP2D | MKN1 |
| 1 | A | A | C |
| 2 | A | A | B |
| 3 | B | A | C |
| 4 | C | B | C |
| 5 | B | A | C |
| 6 | B | A | B |
| 7 | B | A | B |
| 8 | A | A | C |
| 9 | A | A | C |
| 10 | B | A | B |
| 11 | A | A | B |
| 12 | A | A | C |
| 13 | B | B | C |
| 14 | A | A | B |
| 15 | B | C | C |
| 16 | C | B | C |
| 17 | B | A | C |
| 18 | C | — | C |

Although the disclosure has been described in connection with specific embodiments, it should be understood that the disclosure as claimed should not be unduly limited to such specific embodiments. Indeed, various modifications of the described modes for carrying out the disclosure are intended and understood by those skilled in the relevant field in which this disclosure resides to be within the scope of the disclosure as represented by the following claims.

All patents and publications mentioned in this specification are herein incorporated by reference to the same extent as if each independent patent and publication was specifically and individually indicated to be incorporated by reference.

What is claimed is:

1. A compound of the Formula I: 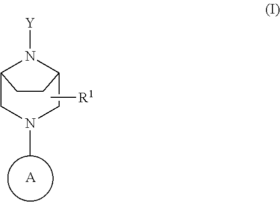
or a pharmaceutically acceptable salt thereof, wherein
Y is CN, C(O)CN, or (C₁-C₄)alkyl optionally substituted with 1 to 3 groups with halo, CN, OH, O(C₁-C₄)alkyl, (C₂-C₄)alkenyl, (C₂-C₄)alkynyl, —COR^{a′}, and —C(O)OR^{a′};
R¹ is hydrogen, halo, OH, (C₁-C₄)alkyl, (C₁-C₄)haloalkyl, (C₁-C₄)hydroxyalkyl, —CHO, —C(O)OR^{b′}, —C(O)ONR^{a′}R^{b′} or a 5- to 6-membered heteroaryl optionally substituted with 1 to 3 groups selected from halo, (C₁-C₄)alkyl, (C₁-C₄)alkoxy, halo(C₁-C₄)alkoxy, and cyano;
R^{a′} and R^{b′} are each independently selected from hydrogen and (C₁-C₄)alkyl, or R^{a′} and R^{b′} are taken together with the nitrogen they attached to form an optionally substituted heterocyclyl; and
\textcircled{A} is optionally substituted heteroaryl.

2. The compound of claim 1, or a pharmaceutically acceptable salt thereof, wherein the compound is of the Formula II: 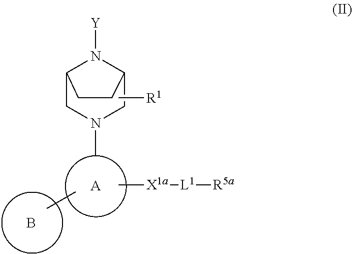
wherein
X^{1a} is selected from S, O, or —NR^{a};
L¹ is optionally substituted (C₁-C₆)alkylene;
R^{5a} is selected from 5- to 10-membered heterocyclyl and —NR′R″;
R′ and R″ are each independently selected from hydrogen and optionally substituted (C₁-C₄)alkyl, or R′ and R″ are taken together with the nitrogen they attached to form an optionally substituted heterocyclyl; and
\textcircled{B} is an optionally substituted aryl or optionally substituted 5- to 10-membered heteroaryl.

3. The compound of claim 1 or 2, or a pharmaceutically acceptable salt thereof, wherein the compound is of the Formula III: 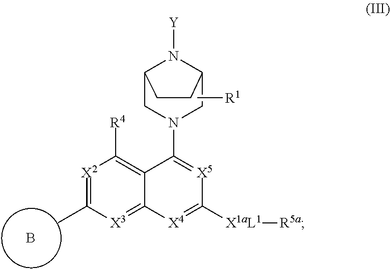
or a pharmaceutically acceptable salt thereof, wherein
X², X³, X⁴, X⁵ are each independently selected from N and CR³; and
R³ and R⁴ are independently selected from hydrogen, (C₁-C₄)alkyl, (C₁-C₄)haloalkyl, (C₁-C₄)alkoxy, deuterated(C₁-C₄)alkoxy, (C₁-C₄)haloalkoxy, (C₁-C₄)alkynyl, (C₁-C₄)alkenyl, halo, (C₃-C₆)cycloalkyl, —O(C₃-C₆)cycloalkyl, cyano, NH₂, —NH(C₁-C₄)alkyl, —N[(C₁-C₄)alkyl]₂, —P(O)[(C₁-C₄)alkyl]₂, and —S(C₁-C₄)alkyl, wherein said (C₃-C₆)cycloalkyl and said (C₃-C₆)cycloalkyl of —O(C₃-C₆)cycloalkyl are optionally substituted with 1 to 3 groups selected from halo, (C₁-C₄)alkyl, (C₁-C₄)alkoxy, halo(C₁-C₄)alkoxy, and cyano.

4. The compound of claim 3, or a pharmaceutically acceptable salt thereof, wherein X², X⁴ and X⁵ is N; and X³ is CF.

5. The compound of claim 3, or a pharmaceutically acceptable salt thereof, wherein X² is CR³, X³ is CF, and X⁴ and X⁵ is N.

6. The compound of claim 3, or a pharmaceutically acceptable salt thereof, wherein X² is CR³ and X³, X⁴, and X⁵ is N.

7. The compound of claim 3, or a pharmaceutically acceptable salt thereof, wherein X², X³, X⁴ and X⁵ is N.

8. The compound of claim 3, or a pharmaceutically acceptable salt thereof, wherein X², X³, and X⁴ is N; and X⁵ is CR³.

9. The compound of claim 3, or a pharmaceutically acceptable salt thereof, wherein X², X³ and X⁵ is N; and X⁴ is CR³.

10. The compound of claim 3, or a pharmaceutically acceptable salt thereof, wherein X² and X⁵ is N, X³ is CF, and X⁴ is CR³.

11. The compound of claim 3, or a pharmaceutically acceptable salt thereof, wherein X² and X⁴ is N, X³ is CF, and X⁵ is CR³.

12. The compound of any one of claims 3 to 11, or a pharmaceutically acceptable salt thereof, wherein R³ is selected from hydrogen, F, Cl, CN, methyl, CF₃, ethyl, and cyclopropyl.

13. The compound of any one of claims 3 to 12, or a pharmaceutically acceptable salt thereof, wherein X¹ is oxygen.

14. The compound of any one of claims 3 to 12, or a pharmaceutically acceptable salt thereof, wherein X¹ is sulfur.

15. The compound of any one of claims 3 to 12, or a pharmaceutically acceptable salt thereof, wherein X¹ is NMe.

16. The compound of any one of claims 3 to 15, or a pharmaceutically acceptable salt thereof, wherein R⁴ is selected from hydrogen, F, Me, CF₃, CHF₂, cyclopropyl, OMe, OEt, OiPr, OCD₃, OCF₃, 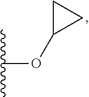 [SMILES: *OC1CC1]
NH₂, NHMe, NMe₂, and SMe.

17. The compound of any one of claims 3 to 16, or a pharmaceutically acceptable salt thereof, wherein L¹ is selected from —CH₂—, —CH₂CH₂—, —CH₂CH₂CH₂—, —CD₂-, 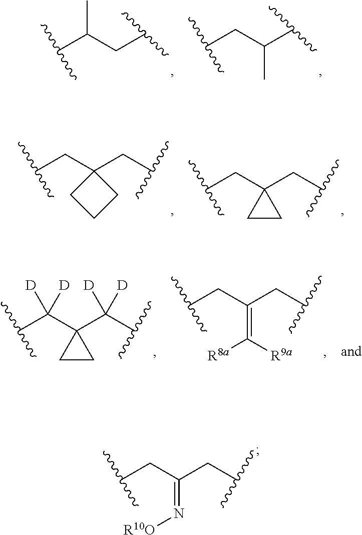
R^{8a} and R^{9a} are each independently selected from hydrogen, halo, CN, (C₁-C₄)alkyl, (C₃-C₆)cycloalkyl, aryl, heteroaryl or R^{8a} and R^{9a} are taken together with the carbon they attached to form cycloalkyl or heterocyclyl; and
R¹⁰ is selected from hydrogen, (C₁-C₄)alkyl, (C₃-C₆)cycloalkyl, aryl, heteroaryl.

18. The compound of any one of claims 3 to 17, or a pharmaceutically acceptable salt thereof, wherein R^{5a} is selected from

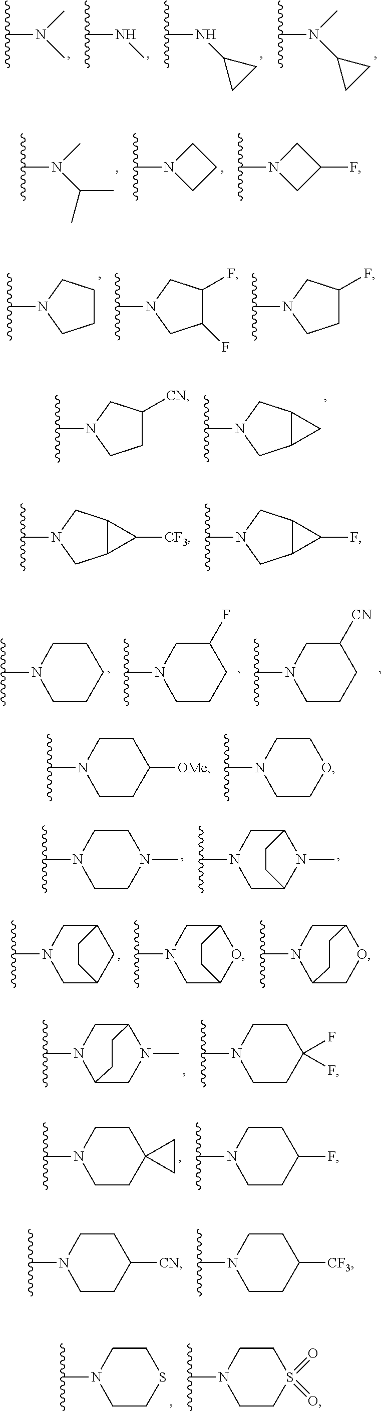

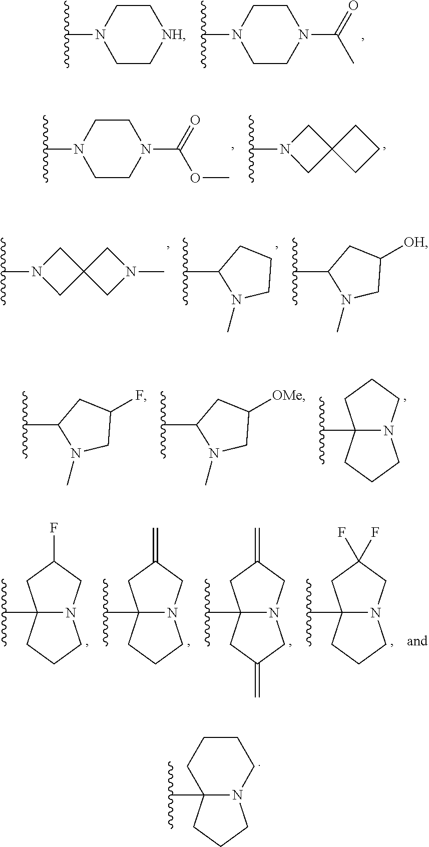

19. The compound of any one of claims 3 to 18, or a pharmaceutically acceptable salt thereof, wherein R³ is selected from

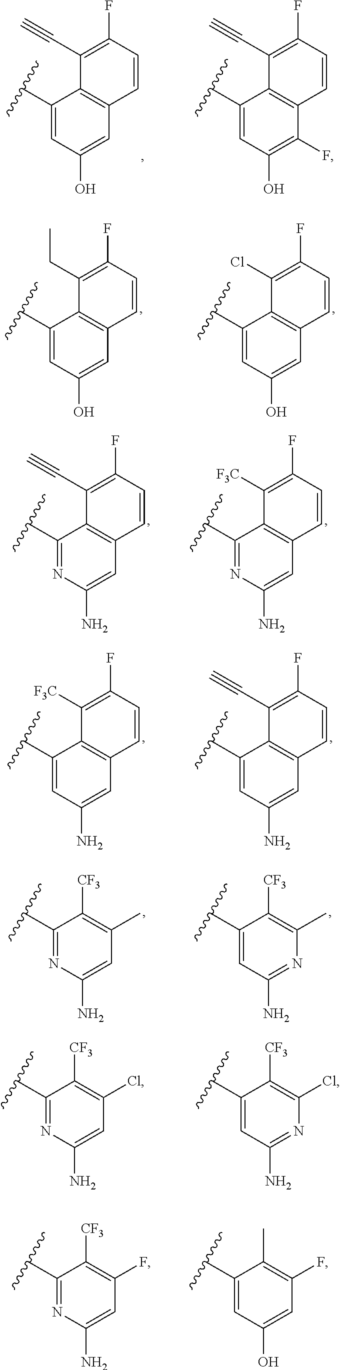

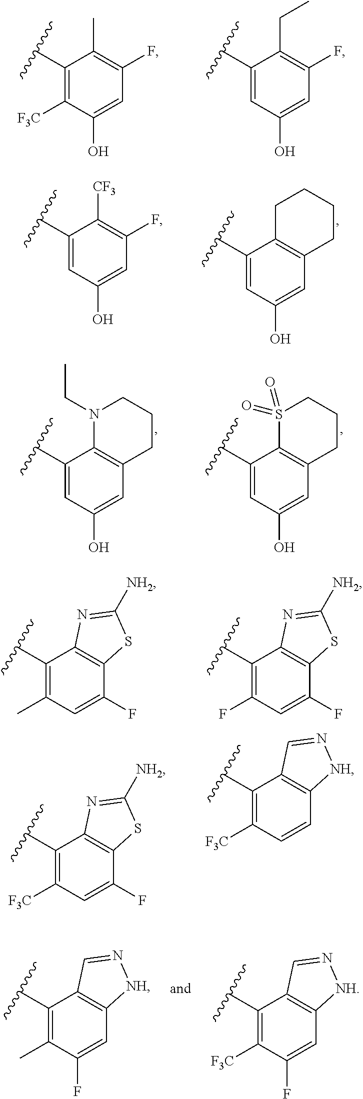

20. The compound of any one of claims 1 to 3, or a pharmaceutically acceptable salt thereof, wherein the compound is of the Formula III^{a}: 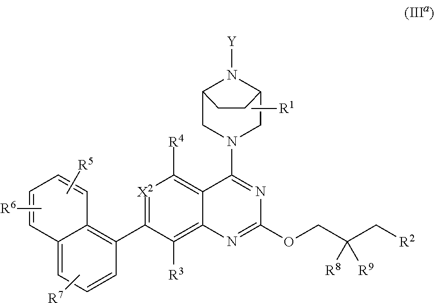
or a pharmaceutically acceptable salt thereof, wherein
X² is CH or N;
R² is a 4- to 6-membered monocyclic heterocyclyl substituted with 1 to 3 groups selected from R^{c} or a 6- to 10-membered bicyclic heterocyclyl optionally substituted with 1 to 3 groups selected from R^{d};
R⁵ is (C₂-C₄)alkynyl;
R⁶ is hydrogen or halo;
R⁷ is hydrogen or OH;
R⁸ and R⁹ are taken together with the carbon they attached to form 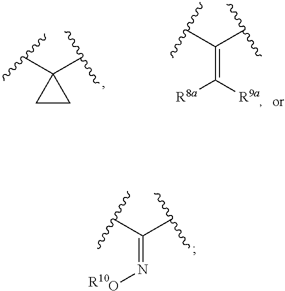
R^{8a} and R^{9a} are each independently selected from hydrogen, halo, (C₁-C₄)alkyl, (C₁-C₄)haloalkyl, (C₁-C₄)alkoxy, deuterated(C₁-C₄)alkoxy, and (C₁-C₄)haloalkoxy; or R^{8a} and R^{9a} are taken together with the carbon they are attached to form (C₃-C₆)cycloalkyl or 4- to 8-membered monocyclic heterocyclyl, wherein said (C₃-C₆)cycloalkyl and said 4- to 8-membered monocyclic heterocyclyl are optionally substituted with 1 to 3 groups selected from halo, (C₁-C₄)alkyl, (C₁-C₄)alkoxy, halo(C₁-C₄)alkoxy, and cyano;
R¹⁰ is selected from hydrogen and optionally substituted (C₁-C₄)alkyl;
R^{a} and R^{b} are each independently selected from hydrogen and (C₁-C₄)alkyl; and
R^{c} and R^{d} are each independently selected from halo, (C₁-C₄)alkyl, (C₁-C₄)haloalkyl, (C₁-C₄)alkoxy, (C₁-C₄)haloalkoxy, cyano, OH, oxo, —C(O)OR^{a}, —C(O)R^{a}, —SO₂R^{a}, —S(O)R^{a}, —SO₂NR^{a}R^{b}, —NR^{a}C(O)R^{b}, —NR^{a}SO₂R^{b}, —NR^{a}R^{b}, and NO.

21. The compound of claim 20, or a pharmaceutically acceptable salt thereof, wherein the compound is of the Formula IV: 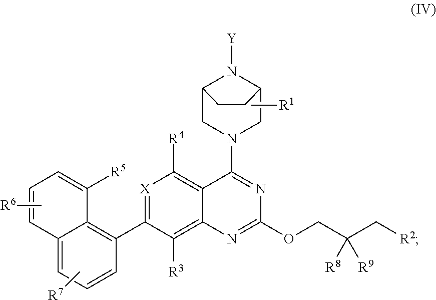
or a pharmaceutically acceptable salt thereof.

22. The compound of claim 20, or a pharmaceutically acceptable salt thereof, wherein the compound the compound is of the Formula V: 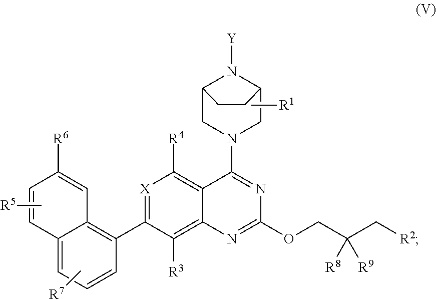
or a pharmaceutically acceptable salt thereof.

23. The compound of claim 20, or a pharmaceutically acceptable salt thereof, wherein the compound is of the Formula VI: 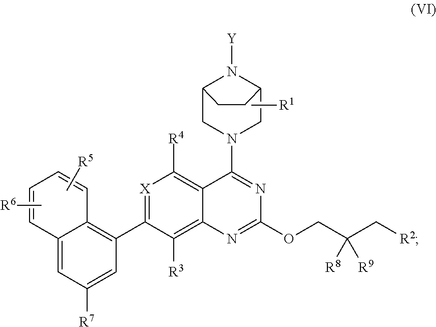
or a pharmaceutically acceptable salt thereof.

24. The compound of claim 20, or a pharmaceutically acceptable salt thereof, wherein the compound is of the Formula VII: 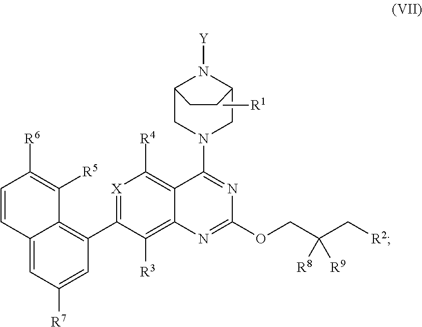
or a pharmaceutically acceptable salt thereof.

25. The compound of any one of claims 1 to 24, or a pharmaceutically acceptable salt thereof, wherein R¹ is hydrogen.

26. The compound of any one of claims 20 to 25, or a pharmaceutically acceptable salt thereof, wherein X is N.

27. The compound of any one of claims 20 to 26, or a pharmaceutically acceptable salt thereof, wherein R³ is halo.

28. The compound of any one of claims 20 to 27, or a pharmaceutically acceptable salt thereof, wherein R³ is fluoro.

29. The compound of any one of claims 20 to 28, or a pharmaceutically acceptable salt thereof, wherein R⁵ is (C₂)alkynyl.

30. The compound of any one of claims 20 to 29, or a pharmaceutically acceptable salt thereof, wherein R⁶ is halo.

31. The compound of any one of claims 20 to 30, or a pharmaceutically acceptable salt thereof, wherein R⁶ is fluoro.

32. The compound of any one of claims 20 to 31, or a pharmaceutically acceptable salt thereof, wherein R⁷ is OH.

33. The compound of any one of claims 20 to 32, or a pharmaceutically acceptable salt thereof, wherein R⁴ is selected from hydrogen, (C₁-C₄)alkoxy, deuterated(C₁-C₄)alkoxy, —N[(C₁-C₄)alkyl]₂, halo, (C₃-C₆)cycloalkyl, (C₁-C₄)haloalkoxy, (C₁-C₄)alkyl, and NH₂.

34. The compound of any one of claims 20 to 33, or a pharmaceutically acceptable salt thereof, wherein R⁴ is selected from hydrogen, methyl, methoxy, isopropoxy, OCD₃, OCDF₂, OCHF₂, —N(CH₃)₂, NH₂, chloro, and cyclopropyl.

35. The compound of any one of claims 20 to 34, or a pharmaceutically acceptable salt thereof, wherein R⁴ is selected from methoxy and OCD₃.

36. The compound of any one of claims 20 to 35, or a pharmaceutically acceptable salt thereof, wherein R² is a 4- to 6-membered nitrogen containing monocyclic heterocyclyl substituted with 1 to 3 groups selected from R^{c} or a 7- to 10-membered nitrogen containing fused or spiro bicyclic heterocyclyl optionally substituted with 1 to 3 groups selected from R^{d}.

37. The compound of any one of claims 20 to 36, or a pharmaceutically acceptable salt thereof, wherein R² is azetidinyl, piperidinyl, morpholinyl, or pyrrolidinyl, each of which being substituted with 1 to 3 groups selected from R^{c} or R² is 3-azabicyclo[3.1.0]hexanyl, 2-azabicyclo[3.1.0]hexanyl, 1,4-dioxa-8-azaspiro[4.5]decanyl, hexahydro-1H-pyrrolizinyl, or 1,2,3,6-tetrahydropyridinyl, each of which being optionally substituted with 1 to 3 groups selected from R^{d}.

38. The compound of any one of claims 20 to 37, or a pharmaceutically acceptable salt thereof, wherein R² is selected from 3-azabicyclo[3.1.0]hexanyl, piperidinyl, and hexahydro-1H-pyrrolizinyl, wherein said piperidinyl is optionally substituted with 1 to 3 groups selected from R^{c} and wherein said 3-azabicyclo[3.1.0]hexanyl and said hexahydro-1H-pyrrolizinyl are each optionally substituted with 1 to 3 groups selected from R^{d}.

39. The compound of any one of claims 20 to 38, or a pharmaceutically acceptable salt thereof, wherein R^{c} and R^{d} are each independently selected from halo, cyano, (C₁-C₄)haloalkyl, (C₁-C₄)alkoxy, (C₁-C₄)alkyl, (C₁-C₄)haloalkoxy, —S(O)R^{a}, and —SO₂NR^{a}R^{b}.

40. The compound of any one of claims 20 to 39, or a pharmaceutically acceptable salt thereof, wherein R^{c} and R^{d} are each independently selected from fluoro, cyano, CF₃, methoxy, isopropyl, OCF₃, —S(O)CH₃, and —SO₂N(CH₃)².

41. The compound of any one of claims 20 to 40, or a pharmaceutically acceptable salt thereof, wherein R^{c} and R^{d} are each fluoro.

42. The compound of any one of claims 20 to 41, or a pharmaceutically acceptable salt thereof, wherein R⁸ and R⁹ are taken together to form

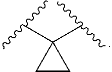 [SMILES: *C1(*)CC1]

43. The compound of any one of claims 1 to 42, or a pharmaceutically acceptable salt thereof, wherein Y is selected from CN, C(O)CN, or (C₁-C₄)alkyl substituted with halo, CN, OH, O(C₁-C₄)alkyl, —COR^{a′}, or —C(O)OR^{a′}.

44. The compound of any one of claims 1 to 43, or a pharmaceutically acceptable salt thereof, wherein Y is selected from CN, C(O)CN, CH₂CN, (CH₂)₂CN, CH₂CF₃, (CH₂)₂CH₃, (CH₂)₂OH, (CH₂)₃OH, CH₂C(O)CH₃, CH₂CHCF₂, (CH₂)₃CN, CH(CH₃)(CH₂)₂CN, (CH₂)₂C(O)OH, (CH₂)₂CH₂F, and CH₂C(O)OH.

45. The compound of claim 1, wherein the compound is selected from any one of the following: 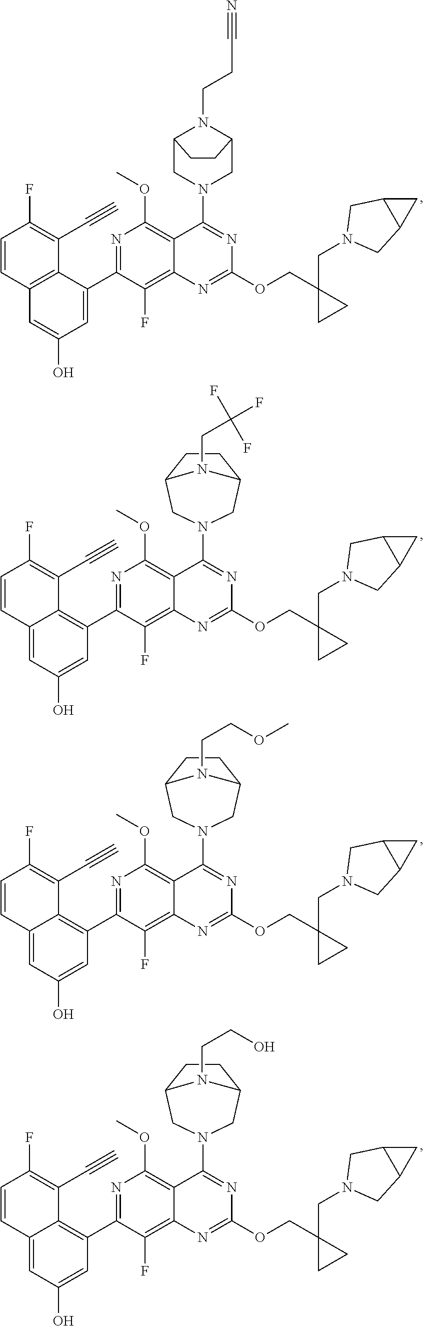 [SMILES: C#Cc1c(F)ccc2cc(O)cc(-c3nc(OC)c4c(N5CC6CCC(C5)N6CCC#N)nc(OCC5(CN6CC7CC7C6)CC5)nc4c3F)c12 ; C#Cc1c(F)ccc2cc(O)cc(-c3nc(OC)c4c(N5CC6CCC(C5)N6CC(F)(F)F)nc(OCC5(CN6CC7CC7C6)CC5)nc4c3F)c12 ; C#Cc1c(F)ccc2cc(O)cc(-c3nc(OC)c4c(N5CC6CCC(C5)N6CCOC)nc(OCC5(CN6CC7CC7C6)CC5)nc4c3F)c12 ; C#Cc1c(F)ccc2cc(O)cc(-c3nc(OC)c4c(N5CC6CCC(C5)N6CCO)nc(OCC5(CN6CC7CC7C6)CC5)nc4c3F)c12] 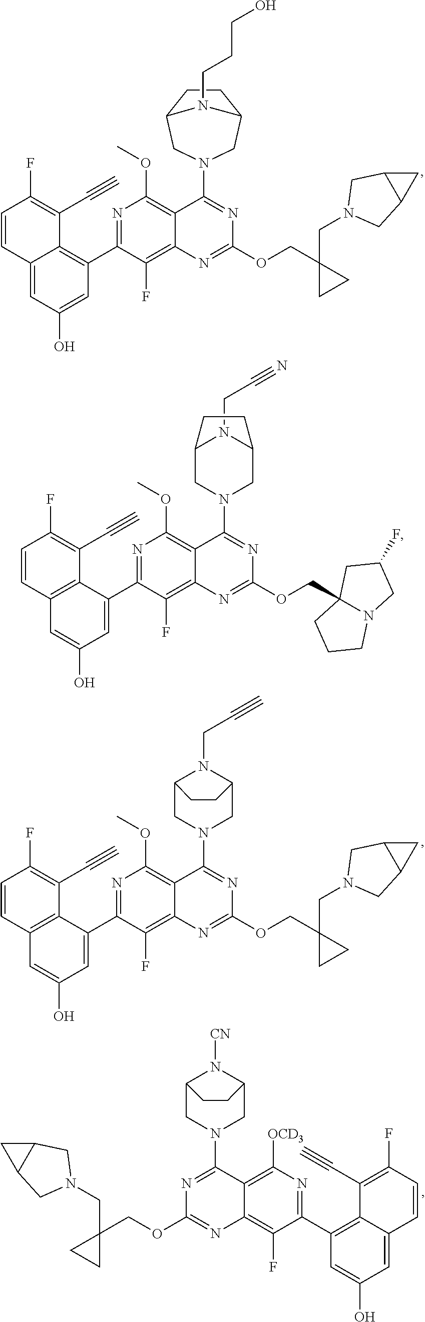 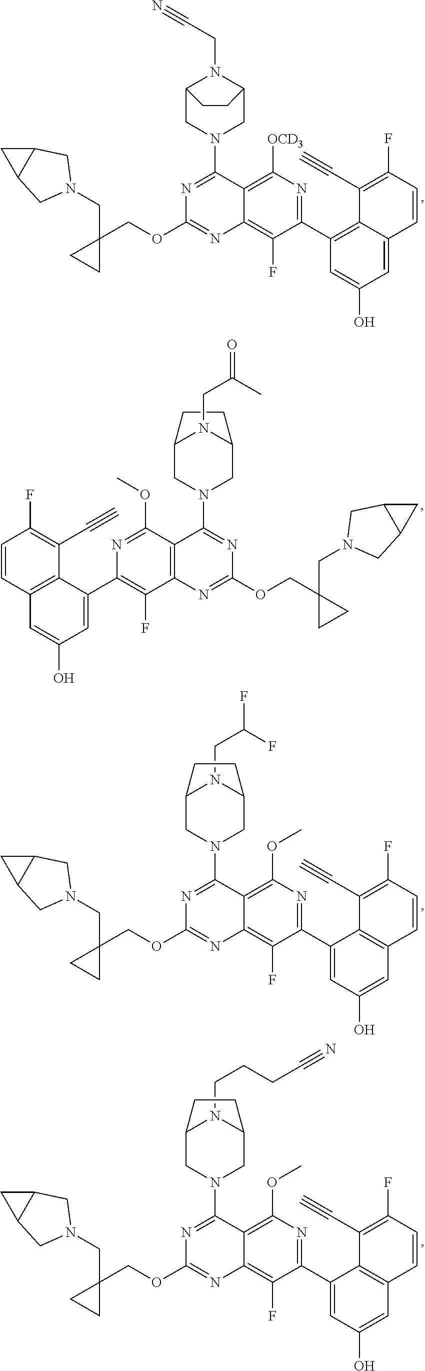 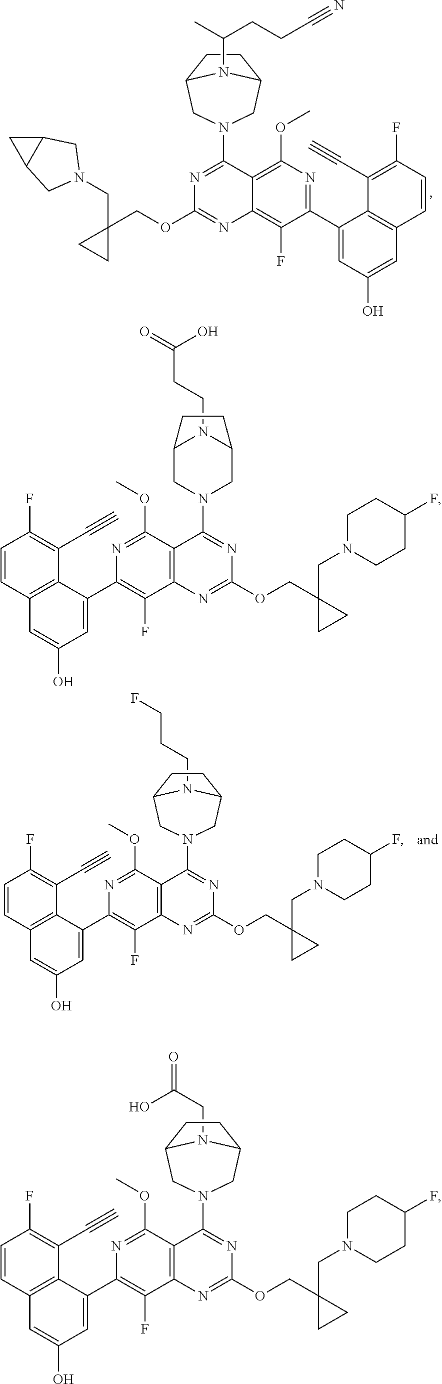 [SMILES: C#Cc1c(F)ccc2cc(O)cc(-c3nc(OC)c4c(N5CC6CCC(C5)N6C(C)CCC#N)nc(OCC5(CN6CC7CC7C6)CC5)nc4c3F)c12 ; C#Cc1c(F)ccc2cc(O)cc(-c3nc(OC)c4c(N5CC6CCC(C5)N6CCC(=O)O)nc(OCC5(CN6CCC(F)CC6)CC5)nc4c3F)c12 ; C#Cc1c(F)ccc2cc(O)cc(-c3nc(OC)c4c(N5CC6CCC(C5)N6CCCF)nc(OCC5(CN6CCC(F)CC6)CC5)nc4c3F)c12 ; C#Cc1c(F)ccc2cc(O)cc(-c3nc(OC)c4c(N5CC6CCC(C5)N6CC(=O)O)nc(OCC5(CN6CCC(F)CC6)CC5)nc4c3F)c12]
or a pharmaceutically acceptable salt of any of the foregoing.

46. A pharmaceutical composition comprising a compound of any one of claims 1 to 45, or a pharmaceutically acceptable salt thereof; and a pharmaceutically acceptable carrier.

47. A method for treating cancer in a subject comprising administering to the subject and effective amount of a compound of any one of claims 1 to 45, or a pharmaceutically acceptable salt thereof, or the pharmaceutically acceptable composition of claim 46.
